SEQUENCE LISTING (8 sequences)

SEQ ID NO: 1 - DNA, 847 bp, Mus musculus
  misc_feature: G0S2
tgcagtcagggctcaggccagtgagccaggaggcggatcccggacaaaagatataaggactctgttcacagagcgctgcagctgcggagtgtccctcccaaagagcagcagctgagggaagaagaacgccaaagccagtctgacgcaaggacgcctgaccttctccagcaagtgctgcctctcttcccactgcaccctaggcccagccacccacagcccagagctcagatggaaagtgtgcaggagctgatccctctggccaaggagatgatggcgcagaagccccgagggaagctagtgaagctatacgtgctgggcagtgtgctggcgctcttcggtgtggtgctcggcctagttgagacggtgtgcagccctttcacagccgccagccgtctgcgcgaccaagaggctgcagtggtggagctgcgggaagcgtgtgaacagcagtccctccacaagcaggccctgctggcaggaggcaaggcacaggaggcgaccctgtgcagccgggccctgtccctccggcagcacgcctcttaaggccggtgactgacagagaagggagacacagatcatgacccaggtgggcaacagagtcacatgctgtttcaaggtgccaccgaatccagaactgaccccacacagatcacctaaggggtctgggactgatttgctgctgtgcagcacgcactgtgatttgccctaggctgtgcgagcaatcaaggagctatcactttgcattagagaaggagacaggctttttatacagttatttttattgttattattattattgcaatgactatcgttttgcattttgaaataaaaaccttttatactctaaaaa

SEQ ID NO: 2 - DNA, 978 bp, Homo sapiens
  misc_feature: G0S2
tggctctgaccgcgctggcctgggccggagagcccaggaggcgtgtctcagagaaaagatataagcggcccccggacgctaaagcggtgccagcggcggagtctccaactgggagagctgcagctgccgagaggaggagaacgctgaggtcggtcggaccaacggacgcgctgaccgctgccaactgcagctcgcgctgcctcctgctcgcgccgtgccactaaggtcattcccgcctccgagagcccagagccgagatggaaacggtccaggagctgatccccctggccaaggagatgatggcccagaagcgcaaggggaagatggtgaagctgtacgtgctgggcagcgtgctggccctcttcggcgtggtgctcggcctgatggagactgtgtgcagccccttcacggccgccagacgtctgcgggaccaggaggcagccgtggcggagctgcaggccgccctggagcgacaggctctccagaagcaagccctgcaggagaaaggcaagcagcaggacacggtcctcggcggccgggccctgtccaaccggcagcacgcctcctaggaactgtgggagaccagcggagtgggagggagacgcagtagacagagacagaccgagagaggaatggagagacagagggggcgcgcgcacaggagcctgactccgctgggagagtgcaggagcacgtgctgttttttatttggacttaacttcagagaaaccgctgacatctagaactgacctaccacaagcatccaccaaaggagtttgggattgagttttgctgctgtgcagcactgcattgtcatgacatttccaacactgtgtgaattatctaaatgcgtctaccattttgcactagggaggaaggataaatgctttttatgttattattattaattattacaatgaccaccattttgcattttgaaataaaaaaactttttataccataaaaaaaaaaaaaaaa

SEQ ID NO: 3 - RNA, 25 nt, Artificial Sequence
  siRNA anti-sense strand against G0S2
aaagggcugcacaccgucucaacua

SEQ ID NO: 4 - RNA, 25 nt, Artificial Sequence
  control siRNA anti-sense strand
cguuggaaucgaguauuccugggaa

SEQ ID NO: 5 - DNA, 64 bp, Artificial Sequence
  DNA fragment for expressing miRNA against G0S2
tgctgttcggtggcaccttgaaacaggttttggccactgactgacctgtttcagtgccaccgaa

SEQ ID NO: 6 - DNA, 64 bp, Artificial Sequence
  DNA fragment for expressing miRNA against G0S2
cctgttcggtggcactgaaacaggtcagtcagtggccaaaacctgtttcaaggtgccaccgaac

SEQ ID NO: 7 - RNA, 25 nt, Artificial Sequence
  siRNA sense strand against Hsd17b4
gagcauuagucauugugaacgacuu

SEQ ID NO: 8 - RNA, 25 nt, Artificial Sequence
  siRNA anti-sense strand against Hsd17b4
aagucguucacaaugacuaaugcuc

(19) United States
(12) Patent Application Publication
(10) Pub. No.: US 2019/0359560 A1
(43) Pub. Date: Nov. 28, 2019

(54) ANTI-INFLAMMATORY DRUG AND USES THEREOF

(71) Applicant: KYUSHU UNIVERSITY, NATIONAL UNIVERSITY CORPORATION, Fukuoka-shi (JP)

(72) Inventors: Shigehiro OHDO, Ogori-shi (JP); Naoya MATSUNAGA, Fukuoka-shi (JP); Akio OJIDA, Kasuya-gun (JP); Naoya SHINDO, Fukuoka-shi (JP)

(73) Assignee: KYUSHU UNIVERSITY, NATIONAL UNIVERSITY CORPORATION, Fukuoka-shi (JP)

(21) Appl. No.: 16/357,768

(22) Filed: Mar. 19, 2019

Related U.S. Application Data
(62) Division of application No. 15/545,831, filed on Dec. 12, 2017, now abandoned.

(30) Foreign Application Priority Data
Jan. 28, 2015 (JP) 2015-014829
Sep. 8, 2015 (JP) 2015-176745

(51) Int. Cl. C07C 279/04; C07C 279/06; A61K 31/155; A61K 31/7105; A61K 31/713; A61K 48/00; C07C 279/18; G01N 33/50; A61P 1/16; C07C 279/16

(52) U.S. Cl. CPC C07C 279/04; C07C 279/06; A61K 31/155; A61K 31/7105; A61K 31/713; C12Q 1/6886; C07C 279/18; G01N 33/5023; A61P 1/16; C07C 279/16; A61K 48/00

(57) ABSTRACT

An anti-inflammatory drug containing a G0/G1 switch 2 (G0S2) inhibitor as an active ingredient is useful as a novel anti-inflammatory drug.

### TECHNICAL FIELD

The present invention relates to an anti-inflammatory drug and uses thereof. More specifically, the present invention relates to an anti-inflammatory drug, a method for screening an anti-inflammatory drug, and a novel compound. This application is a divisional application of U.S. patent application Ser. No. 15/545,831, filed Dec. 12, 2017, which is a National Stage Application of International Patent Application No. PCT/JP2016/052470, filed Jan. 28, 2016, which claims the benefit of Japanese Patent Application No. 2015-14829 filed on Jan. 28, 2015 and Japanese Patent Application No. 2015-176745 filed on Sep. 8, 2015, the contents of which are incorporated herein by reference.

### BACKGROUND ART

Inflammation is one of the symptoms found in various diseases. Currently, many pharmaceutical products that suppress inflammation are available, but there are still diseases with high unmet medical needs among inflammation-related diseases. The inflammation-related disease may be, for example, cancer. Cancer is one of the refractory diseases accompanied by inflammation. Cancer is the leading cause of death in Japan, and the number of deaths due to cancer has been increasing year by year all over the world, which is thus regarded as a problem.

Cancer is found in many organs, but especially hepatoma (hepatocellular cancer) is a cancer type with high mortality rate and recurrence rate. About 90% of the causes of hepatoma are due to viral infection, and hepatoma develops through pathological conditions such as hepatitis starting after viral infection or long-term chronic hepatitis of hepatic cirrhosis (see, for example, NPL 1). Among the pathological conditions, chronic inflammation is known to accelerate the expression of cancer cells resulting from genetic mutation of cells. However, the pathogenic cause of hepatoma, that is, whether hepatoma is due to a virus itself or chronic inflammations has still not yet been clarified (see, for example, NPL 2 and 3). A standard therapy for hepatoma has not currently been established because chemotherapy for hepatoma is likely to result in resistance to anticancer drugs, amongst other reasons. Therefore, there is a need for an innovative diagnostic agent or a novel therapeutic or prophylactic strategy for chronic hepatitis and hepatoma.

Many living organisms have functional systems important for the maintenance of biological homeostasis, which are called circadian clocks. These circadian clocks are constituted by a feedback loop mechanism of transcription and translation by a series of gene groups called clock genes. Variations with a periodicity close to 24 hours (circadian rhythms) are observed in various biological functions, sleep-wake, body temperature, hormone secretion, metabolism, and the like, but it is known that such variations are regulated by the circadian clock mechanism.

In recent years, it has been revealed that the alteration of circadian clock mechanisms is involved in the pathogenesis of various diseases, for example, lifestyle diseases including diabetes, hypertension, gout, and the like (see, for example, NPL 4 and 5). Also with respect to cancer, it has been pointed out that carcinogenesis, properties of cancer cells, and susceptibility to anticancer drugs are affected by circadian clock mechanisms (see, for example, NPL 6).

### CITATION LIST

### Non-Patent Literature

[NPL 1] Bosch F X, et al., Primary liver cancer: worldwide incidence and trends, Gastroenterology, 127, S5-S16, 2004.
[NPL 2] Portolani N, et al., Early and late recurrence after liver resection for hepatocellular carcinoma: prognostic and therapeutic implications, Ann. Surg., 243, 229-235, 2006.
[NPL 3] Freeman A J, et al., Estimating progression to cirrhosis in chronic hepatitis C virus infection, Hepatology, 134, 809-816, 2001.
[NPL 4] Filipski E, et al., Effects of light and food schedules on liver and tumor molecular clocks in mice, J. Nat. Cancer Inst., 97, 507-517, 2005.
[NPL 5] Ohdo S, et al., Molecular basis of chronopharmaceutics., J. Pharm. Sci., 100, 3560-3576, 2011.
[NPL 6] Innominato P F, et al., Chronotherapy and the molecular clock: Clinical implications in oncology., Adv. Drug Deliv. Rev., 62, 979-1001, 2010.

### SUMMARY OF INVENTION

### Technical Problem

However, the relationship between inflammation and circadian clock mechanisms is still unknown in many respects. Therefore, the present invention aims to clarify whether circadian clock mechanisms are involved in inflammation and to provide a novel anti-inflammatory drug.

### Solution to Problem

The present invention includes the following aspects.

[1] An anti-inflammatory drug containing a G0/G1 Switch 2 (G0S2) inhibitor as an active ingredient.

[2] The anti-inflammatory drug according to [1], in which the G0S2 inhibitor is siRNA, shRNA, miRNA, ribozyme or antisense nucleic acid against a G0S2 gene or a hydroxysteroid (1713) dehydrogenase 4 (Hsd17b4) gene.

[3] The anti-inflammatory drug according to [1], in which the G0S2 inhibitor is a specific binding substance for a G0S2 protein or an Hsd17b4 protein.

[4] The anti-inflammatory drug according to [1], in which the G0S2 inhibitor is a compound represented by General Formula (1) or a pharmaceutically acceptable salt thereof, or a solvate thereof:

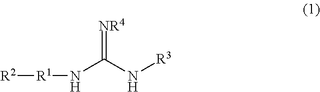

in General Formula (1), R¹ represents a single bond or an alkylene group having 1 to 3 carbon atoms, R² represents

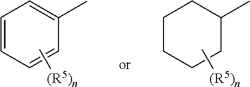

(where R⁵ represents a halogenated alkyl group having 1 to 3 carbon atoms and n represents an integer of 0 to 5), R³ represents an alkyl group having 1 to 15 carbon atoms, and R⁴ represents a hydrogen atom or a carboxylic acid ester group. In the case where n is an integer of 2 or more, a plurality of R⁵s may be the same as or different from each other.

[5] The anti-inflammatory drug according to [4], in which the G0S2 inhibitor has an ability to bind to Hsd17b4.

[6] The anti-inflammatory drug according to any one of [1] to [5], which exhibits an analgesic effect.

[7] A method for screening an anti-inflammatory drug, including a step of measuring an intracellular expression level of a G0S2 gene in the presence of a test substance, and a step of determining that the test substance is an anti-inflammatory drug, in the case where the expression level is decreased as compared to the intracellular expression level of a G0S2 gene in the absence of the test substance.

[8] A method for screening an anti-inflammatory drug, including a step of measuring an activity of an Hsd17b4 protein in the presence of a test substance, and a step of determining that the test substance is an anti-inflammatory drug, in the case where the activity is decreased as compared to the activity of an Hsd17b4 protein in the absence of the test substance.

[9] A compound represented by General Formula (2) or a pharmaceutically acceptable salt thereof, or a solvate thereof:

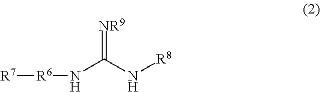

in General Formula (2), R⁶ represents a single bond or an alkylene group having 1 to 3 carbon atoms, R⁷ represents

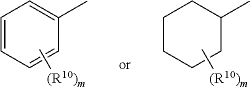

(where R¹⁰ represents a halogenated alkyl group having 1 to 3 carbon atoms and m represents an integer of 0 to 5), R⁸ represents an alkyl group having 1 to 15 carbon atoms, and R⁹ represents a hydrogen atom or a carboxylic acid ester group. In the case where R⁸ is a hexyl group, m is an integer of 1 to 5 and in the case where m is an integer of 2 or more, a plurality of R¹⁰s may be the same as or different from each other.

### Advantageous Effects of Invention

According to the present invention, a novel anti-inflammatory drug can be provided.

### BRIEF DESCRIPTION OF DRAWINGS

FIG. 1 is a schematic diagram showing a pathogenic process of hepatoma.

FIG. 2 is a graph showing an activity of an NF-κB response element in cells to which 0, 12.5, 25 or 50 pmol of siRNA against G0S2 was added in 500 μL of a medium in Experimental Example 2. Each value represents the mean±standard deviation (n=3). As a result of comparison with the results of a non-siRNA-administered group by the Bonferroni-Dunn test, the “**” indicates that there is a significant difference with a risk ratio of less than 1%.

FIG. 3 is a graph showing a binding activity of an NF-κB to an NF-κB response element (NRE) in the liver of a diethylnitrosamine (DEN)-exposed mouse with administration of siRNA against G0S2 or control siRNA in Experimental Example 3. Each value represents the mean±standard deviation (n=3). As a result of comparison with the results of a control siRNA-administered group by the Student's t-test, the “*” indicates that there is a significant difference with a risk ratio of less than 5%.

FIG. 4A is a pathological photomicrograph of the liver of a mouse of a control siRNA-administered group (indicated as “CONTROL siRNA” in the figure) in Experimental Example 3.

FIG. 4B is a photograph of the liver excised from a mouse of a control siRNA-administered group in Experimental Example 3.

FIG. 4C is a pathological photomicrograph of the liver of a mouse of a G0S2 siRNA-administered group (indicated as “G0S2 siRNA” in the figure) in Experimental Example 3.

FIG. 4D is a photograph of a liver excised from a mouse of a G0S2 siRNA-administered group in Experimental Example 3.

FIG. 5 is a graph showing the measurement results of an expression level of Ccl2 mRNA in the liver of a mouse of a G0S2 siRNA-administered group and a mouse of a control siRNA-administered group in Experimental Example 4.

FIG. 6A is a graph showing the results of primary screening of a compound library in Experimental Example 5. The G0S2 promoter (−2030→−1378) activity (relative value) at 24 hours after exposure of cells to 10 μM of each compound is shown as the logarithm with base 2. Each value represents a mean value (n=3).

FIG. 6B is a graph showing the results of secondary screening of a compound library in Experimental Example 5. The ATP activity (relative value) at 24 hours after exposure of cells to 10 μM of each compound is shown as the logarithm with base 2. Each value represents a mean value (n=3).

FIG. 7A is a graph showing the results of screening of compounds in Experimental Example 6. The G0S2 promoter (−2030→+10) activity (relative value) at 24 hours after exposure of cells to 10 μM of each compound is shown as the logarithm with base 2. Each value represents a mean value (n=3).

FIG. 7B shows the logarithm with base 2 of the Bmal1 promoter activity (relative value) at 24 hours after exposure of cells to 10 μM of each compound in Experimental Example 6. Each value represents a mean value (n=3).

FIG. 7C shows the logarithm with base 2 of the Per2 promoter activity (relative value) at 24 hours after exposure of cells to 10 μM of each compound in Experimental Example 6. Each value represents a mean value (n=3). There was no significant difference in Per2 promoter activity (relative value) between the each compound-administered group and the DMSO-administered group.

FIG. 8A is a graph showing the expression level (relative value) of mRNA of G0S2 gene at 24 hours after exposure of cells to 10 μM of Compound Nos. 1 to 3 in Experimental Example 7. Each value represents the mean±standard deviation (n=3). As a result of comparison with the results of the DMSO-administered group by the Bonferroni-Dunn test, the “*” indicates that there is a significant difference with a risk ratio of less than 5%, and the “**” indicates that there is a significant difference with a risk ratio of less than 1%.

FIG. 8B is a graph showing the expression level (relative value) of mRNA of RARα gene at 24 hours after exposure of cells to 10 μM of Compound Nos. 1 to 3 in Experimental Example 7. Each value represents the mean±standard deviation (n=3). As a result of comparison with the results of the DMSO-administered group by the Bonferroni-Dunn test, the “**” indicates that there is a significant difference with a risk ratio of less than 1%.

FIG. 8C is a graph showing the expression level (relative value) of mRNA of Ccl2 gene at 24 hours after exposure of cells to 10 μM of Compound Nos. 1 to 3 in Experimental Example 7. Each value represents the mean±standard deviation (n=3). As a result of comparison with the results of the DMSO-administered group by the Bonferroni-Dunn test, the “*” indicates that there is a significant difference with a risk ratio of less than 5%, and the “**” indicates that there is a significant difference with a risk ratio of less than 1%.

FIG. 8D is a graph showing the expression level (relative value) of mRNA of IL-6 gene at 24 hours after exposure of cells to 10 μM of Compound Nos. 1 to 3 in Experimental Example 7. Each value represents the mean±standard deviation (n=3). As a result of comparison with the results of the DMSO-administered group by the Bonferroni-Dunn test, the “**” indicates that there is a significant difference with a risk ratio of less than 1%.

FIG. 8E is a graph showing a luciferase activity versus a concentration of Compound No. 1 in Experimental Example 8.

FIG. 8F is a graph showing a luciferase activity versus a concentration of Compound No. 2 in Experimental Example 8.

FIG. 9A is a graph showing a transient expression profile of G0S2 mRNA in the liver of a mouse to which Compound No. 2 was administered, in Experimental Example 9. Each value represents the mean±standard deviation (n=3 to 5). As a result of 2-way ANOVA analysis, there was a significant difference with a risk ratio of less than 1% between the control group and the Compound No. 2-administered group. As a result of the Scheffe's test, the “**” indicates that there is a significant difference with a risk ratio of less than 1%.

FIG. 9B is a graph showing a transient expression profile of a G0S2 protein in the liver of a mouse to which Compound No. 2 was administered, in Experimental Example 9. Each value represents the mean±standard deviation (n=3). As a result of comparison with the results of the control group by the Bonferroni-Dunn test, the “**” indicates that there is a significant difference with a risk ratio of less than 1%.

FIG. 10A is a graph showing the expression of Ccl2 mRNA in Hepa1-6 cells at 12 hours after exposure of the cells to Compound No. 2 (10 μM) and LPS (100 ng/mL) in Experimental Example 10. Each value represents the mean±standard deviation (n=3). As a result of comparison with the results of the DMSO-administered group by the Bonferroni-Dunn test, the “**” indicates that there is a significant difference with a risk ratio of less than 1%. As a result of comparison with the results of the LPS-administered group by the Bonferroni-Dunn test, the “##” indicates that there is a significant difference with a risk ratio of less than 1%.

FIG. 10B is a graph showing the expression of Ccl2 mRNA in Hepa1-6 cells at 12 hours after exposure of the cells to Compound No. 2 (10 μM) and TNFα (200 ng/mL) in Experimental Example 10. Each value represents the mean±standard deviation (n=3). As a result of comparison with the results of the DMSO-administered group by the Bonferroni-Dunn test, “**” indicates that there is a significant difference with a risk ratio of less than 1%. As a result of comparison with the results of the LPS-administered group by the Bonferroni-Dunn test, the “##” indicates that there is a significant difference with a risk ratio of less than 1%.

FIG. 10C is a graph showing an effect of Compound No. 2 (5 mg/kg, administered into tail vein) on the expression of Ccl2 mRNA in the liver of a mouse at 6 hours after tail vein administration of LPS, in Experimental Example 10. Each value represents the mean±standard deviation (n=3). As a result of comparison with the results of the DMSO-administered group by the Bonferroni-Dunn test, the “**” indicates that there is a significant difference with a risk ratio of less than 1%. As a result of comparison with the results of the LPS-administered group by the Bonferroni-Dunn test, the “##” indicates that there is a significant difference with a risk ratio of less than 1%.

FIG. 11A is a graph showing the quantitative results of a G0S2 protein in the liver of mice of control group, LPS-administered group, and LPS+Compound No. 2-administered group in Experimental Example 11. Each value represents the mean±standard deviation (n=3 to 5). As a result of the Scheffe's test, the “**” indicates that there is a significant difference with a risk ratio of less than 1%, in comparison with the control group. As a result of the Scheffe's test, the “##” indicates that there is a significant difference with a risk ratio of less than 1%, in comparison with the LPS-administered group.

FIG. 11B is a graph showing the quantitative results of the nuclear abundance of a p65 protein in the liver of mice of control group, LPS-administered group, and LPS+Compound No. 2-administered group in Experimental Example 11. Each value represents the mean±standard deviation (n=3 to 5). As a result of the Scheffe's test, the “**” indicates that there is a significant difference with a risk ratio of less than 1%, in comparison with the control group. As a result of the Scheffe's test, the “##” indicates that there is a significant difference with a risk ratio of less than 1%, in comparison with the LPS-administered group.

FIG. 11C is a graph showing the quantitative results of the expression level of Ccl2 mRNA in the liver of mice of control group, LPS-administered group, and LPS+Compound No. 2-administered group in Experimental Example 11. Each value represents the mean±standard deviation (n=3 to 5). As a result of the Scheffe's test, the “*” indicates that there is a significant difference with a risk ratio of less than 5%, in comparison with the control group. As a result of the Scheffe's test, the “#” indicates that there is a significant difference with a risk ratio of less than 5%, in comparison with the LPS-administered group.

FIG. 12 is a graph showing the time course of PWT after intraperitoneal administration of Compound No. 2 (15 mg/kg) or physiological saline in tumor-bearing mice on day 14, in Experimental Example 12. Each value represents the mean±standard deviation (n=6). As a result of 2-way ANOVA analysis, there was a significant difference with a risk ratio of less than 1% between the control group (0.05% DMSO-physiological saline) and the Compound No. 2-administered group. As a result of the Scheffe's test, the “*” indicates that there is a significant difference with a risk ratio of less than 5% between the control group (0.05% DMSO-physiological saline) and the Compound No. 2-administered group. As a result of the Scheffe's test, the “**” indicates that there is a significant difference with a risk ratio of less than 1% between the control group (0.05% DMSO-physiological saline) and the Compound No. 2-administered group. As a result of the Scheffe's test, the “aa” indicates that there is a significant difference with a risk ratio of less than 1% between before the administration of 0.05% DMSO-physiological saline and after the administration of 0.05% DMSO-physiological saline. As a result of the Scheffe's test, the “bb” indicates that there is a significant difference with a risk ratio of less than 1% between before the administration of Compound No. 2 and after the administration of Compound No. 2.

FIG. 13A is a graph showing the expression level of G0S2 mRNA in Hepa1-6 cells at 24 hours after exposure of the cells to Compound No. 2 and derivatives thereof (10 μM) in Experimental Example 14. Each value represents the mean±standard deviation (n=3). As a result of comparison with the results of the DMSO-administered group by the Bonferroni-Dunn test, the “*” indicates that there is a significant difference with a risk ratio of less than 5%. As a result of comparison with the results of the DMSO-administered group by the Bonferroni-Dunn test, the “**” indicates that there is a significant difference with a risk ratio of less than 1%.

FIG. 13B is a graph showing viability (relative value) of NIH3T3 cells at 24 hours after exposure of the cells to Compound No. 2 and derivatives thereof (10 μM) in Experimental Example 14. Each value represents the mean±standard deviation (n=3). As a result of comparison with the results of the DMSO-administered group by the Bonferroni-Dunn test, the “**” indicates that there is a significant difference with a risk ratio of less than 1%.

FIG. 13C is a graph showing the expression level of G0S2 mRNA in HepG2 cells at 24 hours after exposure of the cells to Compound No. 2 and derivatives thereof (10 μM) in Experimental Example 14. Each value represents the mean±standard deviation (n=3). As a result of comparison with the results of the DMSO-administered group by the Bonferroni-Dunn test, the “**” indicates that there is a significant difference with a risk ratio of less than 1%.

FIG. 13D is a graph showing the expression level of G0S2 mRNA in Hepa1-6 cells at 48 hours after exposure of the cells to Compound No. 2 and derivatives thereof (2.5 or 5 μM) in Experimental Example 14. Each value represents the mean±standard deviation (n=3). As a result of comparison with the results of the DMSO-administered group by the Bonferroni-Dunn test, the “*” indicates that there is a significant difference with a risk ratio of less than 5%. As a result of comparison with the results of the DMSO-administered group by the Bonferroni-Dunn test, the “**” indicates that there is a significant difference with a risk ratio of less than 1%. As a result of comparison with the results of the group to which 5 μM of Compound No. 2 is administered by the Bonferroni-Dunn test, the “##” indicates that there is a significant difference with a risk ratio of less than 1%.

FIG. 14A is a graph showing the results of comparing IC₅₀ between Compound No. 2 and the compound of Example 2 (NS-3-011), which is a derivative of Compound No. 2, in Experimental Example 15. The G0S2 promoter activity (relative value) of O-4000E cells at 24 hours after exposure of the cells to each compound is shown. Each value represents the mean±standard deviation (n=3).

FIG. 14B is a graph showing the expression level of G0S2 mRNA in Hepa1-6 cells at 48 hours after exposure of the cells to 0.5 to 5 μM of Compound No. 2 and the compound of Example 2 (NS-3-011), which is a derivative of Compound No. 2, in Experimental Example 15. Each value represents the mean±standard deviation (n=3). As a result of comparison with the results of the group to which the same dose of Compound No. 2 is administered by the Bonferroni-Dunn test, the “*” indicates that there is a significant difference with a risk ratio of less than 5%. As a result of comparison with the results of the group to which the same dose of Compound No. 2 is administered by the Bonferroni-Dunn test, the “**” indicates that there is a significant difference with a risk ratio of less than 1%.

FIG. 14C is a graph showing the expression level of Ccl2 mRNA in Hepa1-6 cells at 48 hours after exposure of the cells to 0.5 to 5 μM of Compound No. 2 and the compound of Example 2 (NS-3-011), which is a derivative of Compound No. 2, in Experimental Example 15. Each value represents the mean±standard deviation (n=3). As a result of comparison with the results of the group to which the same dose of Compound No. 2 is administered by the Bonferroni-Dunn test, the “*” indicates that there is a significant difference with a risk ratio of less than 5%. As a result of comparison with the results of the group to which the same dose of Compound No. 2 is administered by the Bonferroni-Dunn test, the “**” indicates that there is a significant difference with a risk ratio of less than 1%.

FIG. 14D is a graph showing viability (relative value) of NIH3T3 cells at 24 hours after exposure of the cells to each compound (1 or 5 μM) in Experimental Example 15. Each value represents the mean±standard deviation (n=3 to 6). As a result of comparison with the results of the DMSO-administered group by the Scheffe's test, the “**” indicates that there is a significant difference with a risk ratio of less than 1%.

FIG. 15 is a graph showing the expression levels of G0S2 and Ccl2 mRNAs in control cells and G0S2 knockdown cells in Experimental Example 16. Each value represents the mean±standard deviation (n=3). As a result of comparison with the results of control cells by the Student's t-test, the “**” indicates that there is a significant difference with a risk ratio of less than 1%.

FIG. 16 is a graph showing the expression level of Ccl2 mRNA in the liver of hepatitis model mice administered with the compounds of Example 2, Example 3, and Example 10 in Experimental Example 17. The “VEHICLE” refers to mice without administration of LPS, and the “CONTROL” refers to mice with administration of physiological saline instead of a compound. Each value represents the mean±standard deviation (n=3 to 5). As a result of comparison with the results of the vehicle group by the Scheffe's test, the “*” indicates that there is a significant difference with a risk ratio of less than 5%.

FIG. 17 is a photograph showing the results of acrylamide gel electrophoresis in a pull-down assay for identifying a target protein of the compound of Example 2 in Experimental Example 18.

FIG. 18A is a graph showing the expression level of Ccl2 mRNA in the liver of control mice and NASH model mice with administration of the compound of Example 1 in Experimental Example 19. Each value represents the mean±standard deviation (n=8 to 9). As a result of comparison with the results of the control mice by the Scheffe's test, the “*” indicates that there is a significant difference with a risk ratio of less than 5%.

FIG. 18B is a graph showing the blood drug concentration of the compound of Example 1 in Experimental Example 19. Each value represents the mean±standard deviation (n=3 to 5). As a result of comparison with the results of control cells by the Student's t-test, the “P=0.14” indicates that there is a significant difference with a risk ratio of less than 14%.

FIG. 18C is a graph showing the expression level of αSMA mRNA in the liver of control mice and NASH model mice with administration of the compound of Example 1 in Experimental Example 19. Each value represents the mean±standard deviation (n=8 to 9). As a result of comparison with the results of the control mice by the Scheffe's test, the “*” indicates that there is a significant difference with a risk ratio of less than 5%.

FIG. 18D is a graph showing the expression level of collagen type I α2 (Col1α2) mRNA in the liver of control mice and NASH model mice with administration of the compound of Example 1 in Experimental Example 19. Each value represents the mean±standard deviation (n=8 to 9). As a result of comparison with the results of the control mice by the Scheffe's test, the “*” indicates that there is a significant difference with a risk ratio of less than 5%.

FIG. 18E is a graph showing the expression level of collagen type I al (Col1α1) mRNA in the liver of control mice and NASH model mice with administration of the compound of Example 1 in Experimental Example 19. Each value represents the mean±standard deviation (n=8 to 9). As a result of comparison with the results of the control mice by the Scheffe's test, the “P=0.0673” indicates that there is a significant difference with a risk ratio of less than 6.73%.

FIG. 19 is a graph showing the blood drug concentration of the compound of Example 1 in Experimental Example 20. Each value represents the mean±standard deviation (n=3 to 10). As a result of comparison with the results of the control mice by the Student's t-test, the “P=0.0522” indicates that there is a significant difference with a risk ratio of less than 5.22%.

FIG. 20 is a graph showing the blood drug concentration of the compound of Example 1 in Experimental Example 21. Each value represents the mean±standard deviation (n=5).

FIG. 21A is a graph showing an effect of the compound of Example 10 on the serum ALT activity of a DEN-induced hepatitis/hepatoma mouse model in Experimental Example 22. The results after 8 weeks from the start of administration of DEN in drinking water are shown. Each value represents the mean±standard deviation (n=8 to 10). As a result of comparison with the results of the control mice by the Scheffe's test, the “**” indicates that there is a significant difference with a risk ratio of less than 1%. In addition, as a result of comparison with the results of the DEN-administered group by the Scheffe's test, the “$” indicates that there is a significant difference with a risk ratio of less than 5%.

FIG. 21B is a graph showing an effect of the compound of Example 10 on the serum ALT activity of a DEN-induced hepatitis/hepatoma mouse model in Experimental Example 22. The results after 12 weeks from the start of administration of DEN in drinking water are shown. Each value represents the mean±standard deviation (n=7 to 8). As a result of comparison with the results of the control mice by the Scheffe's test, “**” and “##” indicate that there is a significant difference with a risk ratio of less than 1%. In addition, as a result of comparison with the results of the DEN-administered group by the Scheffe's test, the “$$” indicates that there is a significant difference with a risk ratio of less than 1%.

FIG. 22A is a graph showing the results of knockdown of the Hsd17b4 gene using siRNA against Hsd17b4 in Experimental Example 23. Each value represents the mean±standard deviation (n=3). As a result of comparison with the results of the control siRNA-administered group by the Student's t-test, the “**” indicates that there is a significant difference with a risk ratio of less than 1%.

FIG. 22B is a graph showing the results of quantifying the expression level of the G0S2 gene by real-time PCR following the transfection of cells with siRNA against Hsd17b4 or control siRNA in the presence or absence (indicated as “DMSO” in the figure) of Compound No. 2 in Experimental Example 23. Each value represents the mean±standard deviation (n=3). As a result of comparison with the results in the absence of Compound No. 2 by the Student's t-test, the “*” indicates that there is a significant difference with a risk ratio of less than 5%.

FIG. 23A is a graph showing the results of quantifying the expression level of the Hsd17b4 gene by real-time PCR following the transfection of cells with an expression vector of Hsd17b4 (Hsd17b4-pcDNA3.1) in Experimental Example 24. Each value represents the mean±standard deviation (n=3). As a result of comparison with the results of transfection of cells with an empty vector (pcDNA 3.1) by the Student's t-test, the “**” indicates that there is a significant difference with a risk ratio of less than 1%.

FIG. 23B is a graph showing the results of quantifying the expression level of the G0S2 gene by real-time PCR following the transfection of cells with an expression vector of Hsd17b4 (Hsd17b4-pcDNA3.1) in Experimental Example 24. Each value represents the mean±standard deviation (n=3). As a result of comparison with the results of transfection of cells with an empty vector (pcDNA 3.1) by the Student's t-test, the “**” indicates that there is a significant difference with a risk ratio of less than 1%.

FIG. 24 is a graph showing the results of measuring a luciferase activity in the presence or absence (indicated as “DMSO” in the figure) of Compound No. 2, using cells stably expressing an expression vector in which cDNA of luciferase is ligated to the downstream side of a transcriptional activity regulatory region of G0S2, in Experimental Example 25. Each value represents the mean±standard deviation (n=3). As a result of comparison with the results in the absence of Compound No. 2 by the Student's t-test, the “**” indicates that there is a significant difference with a risk ratio of less than 1%.

FIG. 25A is a diagram showing the structure of a G0S2 transcriptional activity regulatory region.

FIG. 25B is a graph showing the results of measuring a luciferase activity of a cell co-expressing an expression vector in which cDNA of luciferase is ligated to the downstream side of a transcriptional activity regulatory region of G0S2 and a STAT5 expression vector or empty vector (pcDNA 3.1, control) in Experimental Example 26. Each value represents the mean±standard deviation (n=3). As a result of comparison with the control by the Student's t-test, the “**” indicates that there is a significant difference with a risk ratio of less than 1%.

FIG. 26 is a graph showing the results of Western blotting measuring the nuclear abundance of a STAT5 protein in cells cultured in a medium with addition of Compound No. 2 in Experimental Example 27. Each value represents the mean±standard deviation (n=3). As a result of comparison with the control or the 2.5 μM-added group by the Student's t-test, the “**” indicates that there is a significant difference with a risk ratio of less than 1%.

FIG. 27A is a graph showing the results of measuring a drug concentration in the blood of a mouse with oral administration of Compound No. 2 in Experimental Example 28.

FIG. 27B is a graph showing the results of measuring a drug concentration in the liver of a mouse with oral administration of Compound No. 2 in Experimental Example 28.

FIG. 28A is a representative photograph of the liver of a mouse of the control group in Experimental Example 29.

FIG. 28B is a representative photograph of the liver of a mouse of the group with administration of DEN in drinking water in Experimental Example 29.

FIG. 28C is a representative photograph of the liver of a mouse of the group with administration of DEN and the compound of Example 10 in Experimental Example 29.

### DESCRIPTION OF EMBODIMENTS

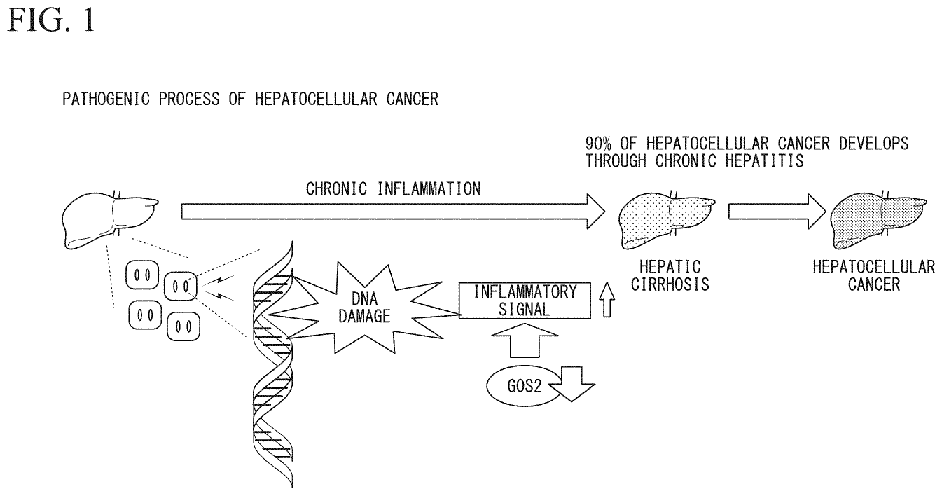

FIG. 1 is a schematic diagram showing a pathogenic process of hepatoma as an example of an inflammation-related disease. About 90% of hepatomas develop through pathological conditions of long-term chronic hepatitis such as hepatitis or hepatic cirrhosis. The influence of G0S2 and the inflammatory signal in the process shown in FIG. 1 was clarified by the present inventors this time, details of which will be described later.

By administering diethylnitrosamine (DEN) to drinking water, the process leading to pathogenesis of chronic hepatitis, hepatic cirrhosis, or hepatoma can be reproduced in an animal. This experimental model is a useful experimental system for elucidating the mechanism of hepatocarcinogenesis in humans. The present inventors have analyzed the pathogenic mechanism of hepatitis/hepatoma focusing on a molecular clock mechanism, using a DEN-induced hepatitis/hepatoma mouse model. As a result, the present inventors have found that G0S2 (G0/G1 switch gene 2), which is a cell cycle regulator, is involved in the pathogenesis of hepatitis/hepatoma.

[Anti-Inflammatory Drug]

In one embodiment, the present invention provides an anti-inflammatory drug containing a G0/G1 Switch 2 (G0S2) inhibitor as an active ingredient. Examples of the G0S2 inhibitor include a compound that inhibits the transcription of G0S2, and a substance that inhibits an activity of G0S2.

Examples of inflammation-related diseases to be treated or prevented by the anti-inflammatory drug of the present embodiment include inflammatory diseases such as hepatitis, small intestine inflammation, colitis (IBD), hepatoma, rheumatism, asthma, atherosclerosis, multiple sclerosis, and Helicobacter pylori-related gastritis.

The anti-inflammatory drug of the present embodiment may be siRNA, shRNA, miRNA, ribozyme or antisense nucleic acid against the G0S2 gene (which inhibits the expression of G0S2).

As will be described later in the Examples, the present inventors have revealed that the expression of the G0S2 gene is controlled via the hydroxysteroid (1743) dehydrogenase 4 (Hsd17b4) protein. That is, it was revealed that the Hsd17b4 protein acts upstream of G0S2 gene expression.

Therefore, it can be said that a substance inhibiting the expression of the Hsd17b4 gene is a G0S2 inhibitor. That is, a substance inhibiting the expression of the Hsd17b4 gene can be used as an anti-inflammatory drug.

Therefore, the anti-inflammatory drug of the present embodiment may be siRNA, shRNA, miRNA, ribozyme or antisense nucleic acid against the Hsd17b4 gene.

Small interfering RNA (siRNA) or microRNA (miRNA) is a small molecule double-stranded RNA consisting of 21 to 23 base pairs used for gene silencing by RNA interference. The siRNA or miRNA introduced into a cell binds to an RNA-induced silencing complex (RISC). This complex binds to mRNA having a sequence complementary to siRNA or miRNA and is then cleaved. This leads to inhibition of gene expression in a sequence-specific manner.

The siRNA or miRNA can be prepared by synthesizing sense strand and antisense strand oligonucleotides using a DNA/RNA automatic synthesizer, and denaturing the synthesized oligonucleotides, for example, in an appropriate annealing buffer at about 90° C. to 95° C. for about 1 minute, followed by annealing at 30° C. to 70° C. for about 1 to 8 hours.

Short hairpin RNA (shRNA) is a hairpin type RNA sequence used for gene silencing by RNA interference. The shRNA may be introduced into a cell by a vector and then expressed with a U6 promoter or H1 promoter or may be prepared by synthesizing an oligonucleotide having an shRNA sequence using a DNA/RNA automatic synthesizer and self-annealing of the synthesized oligonucleotide in the same manner as in siRNA. The hairpin structure of the shRNA introduced into a cell is cleaved into siRNA which then binds to an RNA-induced silencing complex (RISC). This complex binds to mRNA having a sequence complementary to siRNA and is then cleaved. This leads to inhibition of gene expression in a sequence-specific manner.

miRNA is a RNA of about 20 to 25 bases in length existing in a cell and is a kind of non-coding RNA (ncRNA) which is thought to have a function of regulating the expression of other genes. In recent years, it has become possible to knockdown a target gene using artificial miRNA.

Artificial miRNA may be expressed from an expression vector in a mammalian cell with a Pol2 promoter. The miRNA introduced into the cell binds to an RNA-induced silencing complex (RISC). This complex binds to mRNA having a sequence complementary to the miRNA and is then cleaved. This leads to inhibition of gene expression in a sequence-specific manner.

Ribozyme refers to any catalytically active RNA molecule. There are ribozymes having a variety of activities, and, among them, studies on ribozymes as enzymes to cleave RNAs have enabled the design of ribozymes that aim at site-specific cleavage of RNAs. There are ribozymes having a size of 400 nucleotides or more, such as the group I intron ribozyme and M1RNA contained in RNase P, and also those called hammerhead and hairpin ribozymes having a size of about 40 nucleotides.

Antisense nucleic acid is a nucleic acid complementary to a target sequence. The antisense nucleic acid is capable of inhibiting the expression of a target gene through the following action mechanisms: the inhibition of transcription initiation by triplex formation; transcription inhibition by hybrid formation with a site where an open loop structure is locally formed by RNA polymerase; transcription inhibition by hybrid formation with RNA during its synthesis; splicing inhibition by hybrid formation at intron-exon junctions; splicing inhibition by hybrid formation with a spliceosome formation site; inhibition of nuclear-cytoplasmic transport by hybrid formation with mRNA; splicing inhibition by hybrid formation with a capping or poly(A)-addition site; inhibition of translation initiation by hybrid formation with a translation initiation factor-binding site; translation inhibition by hybrid formation with a ribosome-binding site in proximity to an initiation codon; inhibition of peptide chain elongation by hybrid formation with a mRNA translation region or polysome binding site; and inhibition of gene expression by hybrid formation with a nucleic acid-protein interaction site.

In the present embodiment, siRNA, shRNA, ribozyme and antisense nucleic acid may contain various chemical modifications in order to improve stability and activity thereof. For example, phosphate residues may be substituted with chemically modified phosphate residues such as phosphorothioate (PS), methyl phosphonate, and phosphorodithionate in order to prevent degradation of siRNA, shRNA, ribozyme or antisense nucleic acid by hydrolases such as nucleases. Further, at least a part of siRNA, shRNA, ribozyme or antisense nucleic acid may be constituted by a nucleic acid analog such as peptide nucleic acid (PNA).

The anti-inflammatory drug of the present embodiment may be a specific binding substance for the G0S2 protein or Hsd17b4 protein. The specific binding substance exerts an anti-inflammatory effect by specifically binding to the G0S2 protein or Hsd17b4 protein and inhibiting the function of the protein.

Examples of the specific binding substance include an antibody, an antibody fragment, an aptamer, and a low-molecular-weight compound. The antibody can be constructed, for example, by immunizing an animal such as a mouse with an antigen. Alternatively, the antibody can be constructed by screening an antibody library such as a phage library.

Examples of the antibody fragment include Fv, Fab, and scFv. The foregoing antibody or antibody fragment may be polyclonal or monoclonal. The foregoing antibody or antibody fragment may be, for example, an antibody or antibody fragment to which a compound such as polyethylene glycol is bound. Binding of polyethylene glycol makes it possible to increase blood stability, for example.

An aptamer is a substance having a specific binding ability to a labeling substance. Examples of the aptamer include a nucleic acid aptamer and a peptide aptamer. The nucleic acid aptamer having a specific binding ability to the G0S2 protein or Hsd17b4 protein can be selected by, for example, a systematic evolution of ligand by exponential enrichment (SELEX) method. The peptide aptamer having a specific binding ability to the G0S2 protein or Hsd17b4 protein can be selected by, for example, a two-hybrid method using yeast.

In addition to the foregoing, the specific binding substance may be, for example, a substance screened from a compound library or the like by means of the bindability to a target substance as an index.

As will be described later in the Examples, it is possible to inhibit the pathogenesis of hepatitis and hepatoma by administering siRNA against G0S2 to a DEN-induced hepatitis/hepatoma model mouse. Therefore, it can be said that the anti-inflammatory drug of the present embodiment is an agent for treating or preventing hepatitis or hepatoma.

The anti-inflammatory drug of the present embodiment may be a compound represented by General Formula (1) or a pharmaceutically acceptable salt thereof, or a solvate thereof.

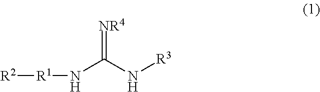

In General Formula (1), R¹ represents a single bond or an alkylene group having 1 to 3 carbon atoms, R² represents

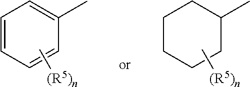

(where R⁵ represents a halogenated alkyl group having 1 to 3 carbon atoms and n represents an integer of 0 to 5), R³ represents an alkyl group having 1 to 15 carbon atoms, and R⁴ represents a hydrogen atom or a carboxylic acid ester group. In the case where n is an integer of 2 or more, a plurality of R⁵s may be the same as or different from each other.

In the anti-inflammatory drug of the present embodiment, the compound represented by General Formula (1) may be a free form, a pharmaceutically acceptable salt, a solvate of a free form, or a solvate of a pharmaceutically acceptable salt.

Examples of the pharmaceutically acceptable salt include a metal salt, an ammonium salt, an organic amine addition salt, and amino acid addition salt. More specific examples of the pharmaceutically acceptable salt include an inorganic acid salt such as hydrochloride, sulfate, hydrobromide, nitrate, phosphate, or hydroiodide; an organic acid salt such as acetate, mesylate, succinate, maleate, fumarate, citrate, tartrate, benzoate, methanesulfonate, 2-hydroxyethanesulfonate, p-toluenesulfonate, trifluoroacetate, propanoate, oxalate, malonate, glutarate, adipate, malate, or mandelate; an alkali metal salt such as sodium salt or potassium salt; an alkaline earth metal salt such as magnesium salt or calcium salt; a metal salt such as aluminum salt or zinc salt; an ammonium salt such as ammonium salt or tetramethylammonium salt; an organic amine addition salt such as morpholine or piperidine addition salt; and an amino acid addition salt such as glycine, phenylalanine, lysine, aspartic acid, or glutamic acid addition salt.

The solvate is not particularly limited as long as it is a pharmaceutically acceptable solvate, and examples thereof include a hydrate and an organic solvate.

The compound represented by General Formula (1) or a pharmaceutically acceptable salt thereof, or a solvate thereof may have a binding ability to Hsd17b4 (Peroxisomal multifunctional enzyme type 2).

As will be described later in the Examples, the present inventors have revealed that the compound represented by General Formula (1) specifically binds to Hsd17b4. Hsd17b4 is a protein known to exist in a peroxisome or the like and is an enzyme having an activity such as β-oxidation of fatty acids. The Uniprot accession number of human Hsd17b4 is P51659 and the Uniprot accession number of mouse Hsd17b4 is P51660.

As will be described later in the Examples, the anti-inflammatory drug of the present embodiment exhibits an analgesic effect. Therefore, it can be said that the anti-inflammatory drug of the present embodiment is an analgesic agent.

[Pharmaceutical Composition]

The anti-inflammatory drug of the present embodiment may be administered per se or in the form of a formulated pharmaceutical composition in admixture with a pharmaceutically acceptable carrier.

The pharmaceutical composition may be formulated into an oral dosage form such as a tablet, capsule, elixir, or microcapsule, or may be formulated into a parenteral dosage form such as an injection, ointment, or patch.

Examples of the pharmaceutically acceptable carrier include a solvent such as sterilized water or physiological saline; a binder such as gelatin, corn starch, tragacanth gum, or gum arabic; an excipient such as crystalline cellulose; and a swelling agent such as corn starch, gelatin, or alginic acid.

The pharmaceutical composition may contain an additive. Examples of the additive include a lubricant such as magnesium stearate; a sweetener such as sucrose, lactose or saccharin; a flavoring agent such as peppermint or Gaultheria adenothrix oil; a stabilizer for benzyl alcohol or phenol; a buffer such as phosphate or sodium acetate; a solubilizer such as benzyl benzoate or benzyl alcohol; an antioxidant; a preservative; a surfactant; and an emulsifier.

The pharmaceutical composition can be formulated by appropriately combining the above-mentioned carrier and additives and mixing the ingredients in a unit dosage form required in generally accepted pharmaceutical practice.

In the case where the pharmaceutical composition is an injection, the solvent for an injection may be, for example, physiological saline, or an isotonic solution containing an adjuvant such as glucose, D-sorbitol, D-mannose, D-mannitol, or sodium chloride. The solvent for an injection may contain an alcohol such as ethanol; a polyalcohol such as propylene glycol or polyethylene glycol; and a nonionic surfactant such as polysorbate 80 (trademark) or HCO-50.

The administration of the anti-inflammatory drug to a patient may be carried out, for example, by a method known to those skilled in the art via an intranasal, transbronchial, intramuscular, transdermal or oral route as well as by intraarterial injection, intravenous injection, or subcutaneous injection.

The dose of the anti-inflammatory drug varies depending on the patient's symptoms and the like. In the case of oral administration, the dose of the anti-inflammatory drug may be, for example, 5 to 20 mg/kg/day. In the case of parenteral administration, the dose varies depending on the subject to be administered, target organ, symptoms, and administration method. For example, in the case of subcutaneous administration, for example, a dose of 10 to 25 mg/kg/day may be administered once. If the dose exceeds the above-specified range, hepatic damage may occur. The lethal dose in the case of intravenous injection is about 25 mg/kg.

[Method for Screening Anti-Inflammatory Drug]

In one embodiment, the present invention provides a method for screening an anti-inflammatory drug, including a step of measuring an intracellular expression level of a G0S2 gene in the presence of a test substance, and a step of determining that the test substance is an anti-inflammatory drug, in the case where the expression level is decreased as compared to the intracellular expression level of a G0S2 gene in the absence of the test substance. As will be described later in the Examples, the anti-inflammatory drug can be screened by the screening method of the present embodiment.

As the test substance, for example, a compound library or the like may be used. The test substance may be added to, for example, a cell culture medium. The method for measuring the expression level of the G0S2 gene is not particularly limited, and may be carried out by, for example, real-time PCR or may be carried out at a protein level by Western blotting, ELISA or the like.

Alternatively, the expression level of the G0S2 gene may be measured by a reporter assay where an expression vector in which a reporter gene such as a luciferase gene or the like has been ligated to the downstream side of the G0S2 promoter is constructed, and cells transfected with the expression vector are used for the assay.

It can be determined that the test substance is an anti-inflammatory drug, in the case where the expression level of a G0S2 gene in the presence of the test substance is decreased as compared to that of the control (in the absence of the test substance).

In one embodiment, the present invention provides a method for screening an anti-inflammatory drug, including a step of measuring an activity of an Hsd17b4 protein in the presence of a test substance, and a step of determining that the test substance is an anti-inflammatory drug, in the case where the activity is decreased as compared to the activity of the Hsd17b4 protein in the absence of the test substance.

As will be described later in the Examples, the present inventors have revealed that the Hsd17b4 protein functions upstream of G0S2. Therefore, it can be said that a test substance decreasing the activity of the Hsd17b4 protein is an inhibitor of G0S2. That is, a test substance decreasing the activity of the Hsd17b4 protein can be said to be an anti-inflammatory drug.

The screening method of the present embodiment may be carried out at a cellular level or may be carried out in vitro using the purified Hsd17b4 protein. As the test substance, the same substances as those described above can be used.

As the activity of the Hsd17b4 protein, a fatty acid β-oxidation activity may be mentioned. The activity of the Hsd17b4 protein may be measured, for example, by measuring a metabolic rate of 2-methyl-branched-chain fatty acids, bile acid intermediates, or very long chain fatty acids. The present inventors have revealed from the computer analysis results that the activity of Hsd17b4 decreases as a result of binding of a compound such as NS-3-011 to the enzyme active site of Hsd17b4. Compounds other than NS-3-011 can also bind to Hsd17b4.

### Other Embodiments

In one embodiment, the present invention provides a method for treating or preventing an inflammation-related disease, including administering siRNA, shRNA, miRNA, ribozyme or antisense nucleic acid against G0S2, or a compound represented by General Formula (1) or a pharmaceutically acceptable salt thereof, or a solvate thereof to a patient or animal in need of treatment.

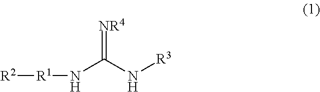

In General Formula (1), R¹ represents a single bond or an alkylene group having 1 to 3 carbon atoms, R² represents

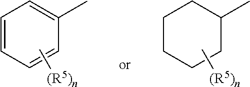

(where R⁵ represents a halogenated alkyl group having 1 to 3 carbon atoms and n represents an integer of 0 to 5), R³ represents an alkyl group having 1 to 15 carbon atoms, and R⁴ represents a hydrogen atom or a carboxylic acid ester group. In the case where n is an integer of 2 or more, a plurality of R⁵s may be the same as or different from each other.

In one embodiment, the present invention provides a method for treating or preventing pain, including administering siRNA, shRNA, miRNA, ribozyme or antisense nucleic acid against G0S2, or a compound represented by General Formula (1) or a pharmaceutically acceptable salt thereof, or a solvate thereof to a patient or animal in need of treatment.

In one embodiment, the present invention provides a method for treating or preventing hepatitis or hepatoma, including administering siRNA, shRNA, miRNA, ribozyme or antisense nucleic acid against G0S2, or a compound represented by General Formula (1) or a pharmaceutically acceptable salt thereof, or a solvate thereof to a patient or animal in need of treatment.

In one embodiment, the present invention provides siRNA, shRNA, miRNA, ribozyme or antisense nucleic acid against G0S2, or a compound represented by General Formula (1) or a pharmaceutically acceptable salt thereof, or a solvate thereof for treating or preventing an inflammation-related disease or for treating or preventing pain.

In one embodiment, the present invention provides uses of siRNA, shRNA, miRNA, ribozyme or antisense nucleic acid against G0S2, or a compound represented by General Formula (1) or a pharmaceutically acceptable salt thereof, or a solvate thereof, in the manufacture of an agent for treating or preventing an inflammation-related disease or an agent for treating or preventing pain.

[Novel Compound]

The present inventors have also found a compound represented by General Formula (2).

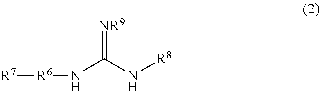

In General Formula (2), R⁶ represents a single bond or an alkylene group having 1 to 3 carbon atoms, R⁷ represents

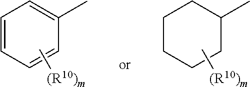

(where R¹⁰ represents a halogenated alkyl group having 1 to 3 carbon atoms and m represents an integer of 0 to 5), R⁸ represents an alkyl group having 1 to 15 carbon atoms, and R⁹ represents a hydrogen atom or a carboxylic acid ester group. In the case where R⁸ is a hexyl group, m is an integer of 1 to 5, and in the case where m is an integer of 2 or more, a plurality of R¹⁰s may be the same as or different from each other.

Like the compound represented by General Formula (1), the compound represented by General Formula (2) may be a free form, a pharmaceutically acceptable salt, a solvate of a free form, or a solvate of a pharmaceutically acceptable salt. The pharmaceutically acceptable salt and solvate are the same as those described above.

As will be described later, the compound of the present embodiment exerts a remarkably high effect of inhibiting the expression of G0S2 as compared to a compound represented by General Formula (3) (Compound No. 2). Therefore, the compound of the present embodiment can be used as, for example, an anti-inflammatory drug.

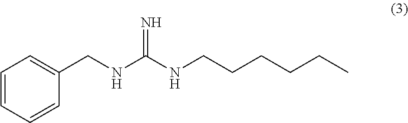

The compound of the present embodiment may be formulated as a pharmaceutical composition in the same manner as in the compound represented by General Formula (1). In this case, the dosage form, administration method and dosage of the pharmaceutical composition are the same as those described above.

### Examples

Next, the present invention will be described in more detail with reference to Experimental Examples, but the present invention is not limited to the following Experimental Examples.

### Experimental Example 1

(Identification of G0S2)

Alteration in circadian clock mechanisms was observed in a DEN-induced hepatitis/hepatoma mouse model. As a result, alteration was observed in the expression rhythm of G0S2 (G0/G1 switch gene 2). The base sequence of mouse G0S2 is set forth in SEQ ID NO: 1 and the base sequence of human G0S2 is set forth in SEQ ID NO: 2.

### Experimental Example 2

(Function analysis of G0S2)

G0S2 in cultured cells was knocked down, followed by investigation of the effect of such knockdown of G0S2 on NF-κB, which is a transcription factor activated by inflammatory signals.

First, a reporter assay using an NF-κB response element was carried out. A cell stably expressing an expression cassette in which a luciferase gene was ligated to the downstream side of the NF-κB response element was constructed in NIH3T3, which is a mouse embryo-derived fibroblast cell line. Subsequently, 0, 12.5, 25 and 50 pmol of siRNA against G0S2 was added to the medium (volume of 500 μL) of this cell, followed by allowing to stand for 48 hours. As the siRNA against G0S2, commercially available siRNA (Life Technologies, Inc., trade name “Stealth siRNA”, catalog number “#1320001”, the base sequence of the antisense strand is set forth in SEQ ID NO: 3) was used.

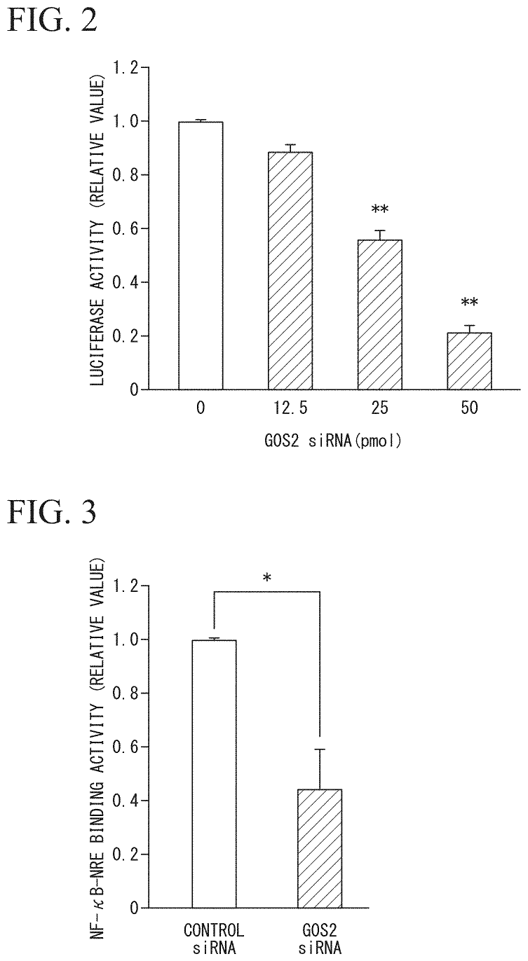

Subsequently, each cell was recovered, disrupted, and allowed to react with a luminescent substrate (trade name “Dual-Luciferase® Reporter Assay System”, manufactured by Promega Corporation) to measure the amount of luminescence, thereby measuring the activity of the NF-κB response element. FIG. 2 is a graph showing the measurement results. As a result, it was revealed that the activity of the NF-κB response element in NIH3T3 cells was decreased by adding siRNA against G0S2 to the medium.

These results revealed that G0S2 is a regulator of a transcriptional activity of an inflammatory signal.

### Experimental Example 3

(Knockdown of G0S2)

Mice of a diethylnitrosamine (DEN)-induced hepatitis/hepatoma mouse model were divided into two groups, one receiving siRNA against G0S2 (G0S2 siRNA-administered group) and the other receiving negative control siRNA (control siRNA-administered group). Administration of siRNA to mice was carried out by tail vein administration of 1.6 nmol/mouse using a nucleic acid transfection reagent (trade name “LipoTrust™ EX Oligo <in vivo>, Hokkaido System Science Co., Ltd.). As the siRNA against G0S2, commercially available siRNA (Life Technologies, Inc., trade name “Stealth siRNA”, catalog number “#1320001”, the base sequence of the antisense strand is set forth in SEQ ID NO: 3) was used. As the control siRNA, a commercially available siRNA (Life Technologies, Inc., the base sequence of the antisense strand is set forth in SEQ ID NO: 4) was used.

DEN was added to the drinking water at a concentration of 80 mg/L from 1 day after the administration of siRNA to mice. The liver was harvested three days after the administration of siRNA to mice and then subjected to a gel shift assay using a DNA fragment containing an NF-κB response element sequence to thereby measure the binding amount between NF-κB and NF-κB response element.

FIG. 3 is a graph showing the measurement results. As a result, it was revealed that administration of siRNA against G0S2 resulted in a decreased binding activity of NF-κB to an NF-κB response element (NRE) in the liver of a mouse hepatitis/hepatoma pathogenesis model.

Subsequently, mice of the same G0S2 siRNA-administered group and control siRNA-administered group as above were prepared. DEN was added to drinking water at a concentration of 80 mg/L from one day after the administration of siRNA to mice, and mice were sacrificed after 14 weeks of breeding and then subjected to analysis. As a result, the onset of hepatoma was inhibited in mice of the G0S2 siRNA-administered group. On the other hand, the onset of hepatoma was observed in mice of the control siRNA-administered group.

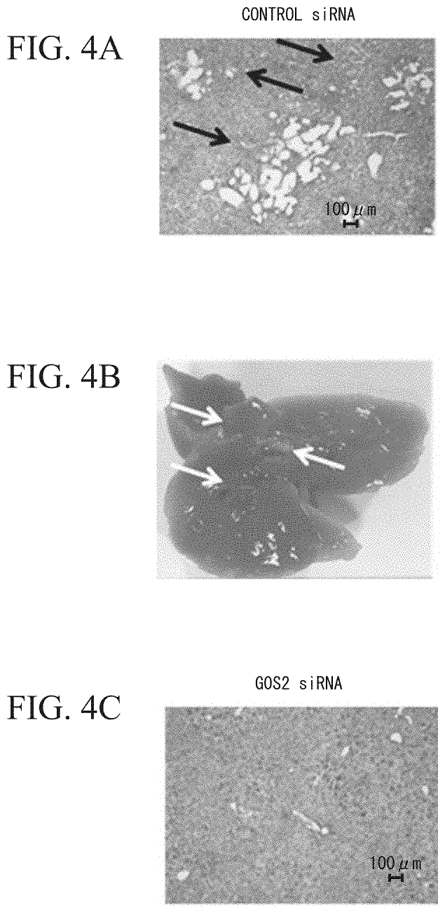

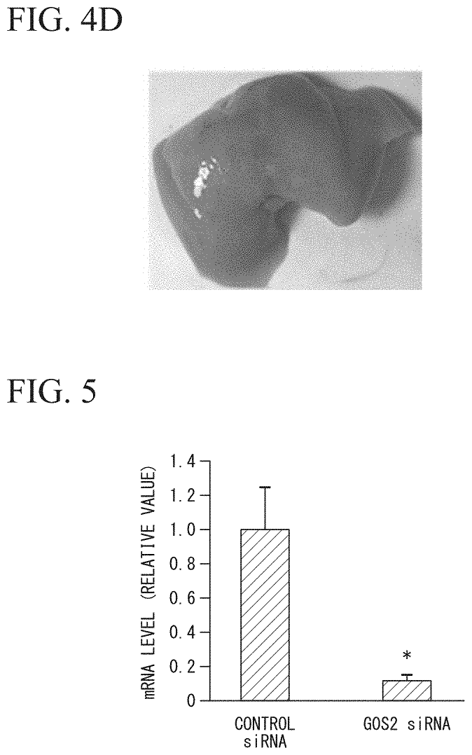

FIG. 4A is a pathological photomicrograph of the liver of the mouse of the control siRNA-administered group (indicated as “CONTROL siRNA” in the figure), and FIG. 4B is a photograph of the liver excised from the mouse of the control siRNA-administered group. The arrows in FIG. 4A indicate cancer cells, and the arrows in FIG. 4B indicate hepatoma developed.

FIG. 4C is a pathological photomicrograph of the liver of the mouse of the G0S2 siRNA-administered group (indicated as “G0S2 siRNA” in the figure), and FIG. 4D is a photograph of the liver excised from the mouse of the G0S2 siRNA-administered group.

### Experimental Example 4

(Study of Expression Level of Ccl2)

The expression level of mRNA of chemokine Ccl2 was measured by a qRT-PCR method in the liver of the mouse of the G0S2 siRNA-administered group and the mouse of the control siRNA-administered group in Experimental Example 3. FIG. 5 is a graph showing the measurement results of the expression level of Ccl2 mRNA in the liver of mouse of each group. The asterisk (*) in the figure means that there is a significant difference with a risk rate of less than 5%.

As a result, it was found that the expression of Ccl2 was significantly inhibited in the mouse of the G0S2 siRNA-administered group (indicated as “G0S2 siRNA” in the figure) as compared to the mouse of the control siRNA-administered group (indicated as “CONTROL siRNA” in the figure).

### Experimental Example 5

(Primary and Secondary Screening Using Compound Library)

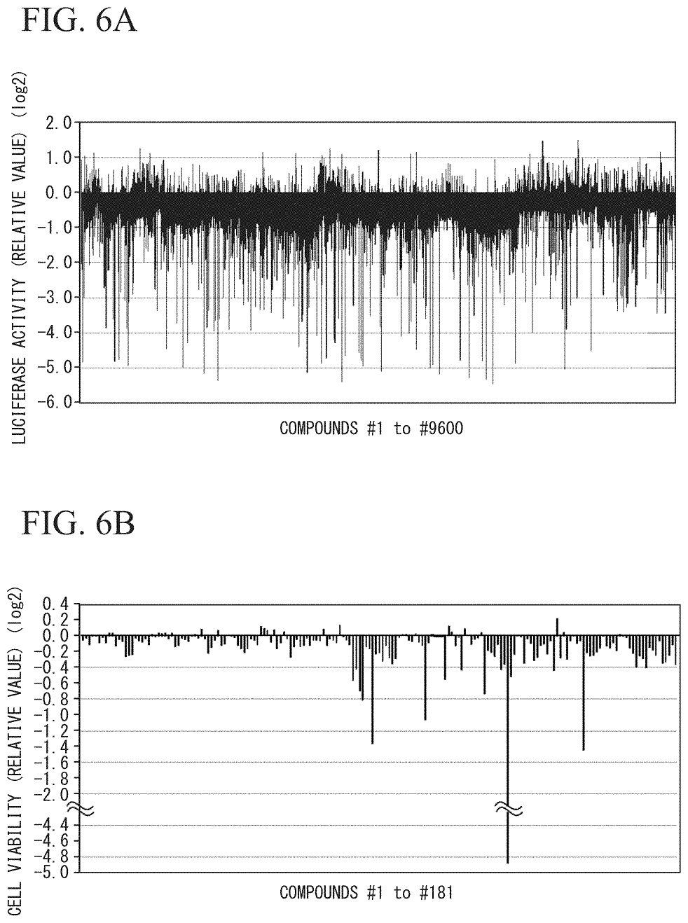

The present inventors have searched for a G0S2 gene transcription inhibitor. In searching, the present inventors have received a compound library consisting of 9600 compounds (Core library) from the Open Innovation Center for Drug Discovery, The University of Tokyo. For this compound library, primary screening was carried out by evaluating an effect of compounds on a luciferase activity using a 384-well plate. The drug efficacy evaluation was studied three times for each compound (final concentration of 10 μM) using a cell line stably expressing an expression cassette in which a luciferase gene was ligated to the downstream side of the G0S2 promoter (−2030→−1378), and compounds which reduced a luciferase activity to less than ¼ were extracted from all studies. As a result, 175 compounds were hit. FIG. 6A shows the results of the primary screening.

Next, secondary screening was carried out by evaluating an effect of these compounds (final concentration of 10 μM) on the viability of the O-4000 E cell line, which is a mouse fibroblast-derived cell, using the intracellular ATP abundance as an index. FIG. 6B shows the results of the secondary screening. As a result, there were very few compounds that significantly reduced the cell viability. Therefore, 50 compounds which do not have an effect on the cell viability and lower the luciferase activity of the O-4000E cell line to less than 1/10 in the primary screening were selected as candidate compounds.

### Experimental Example 6

(Screening by Selectivity for G0S2 Promoter)

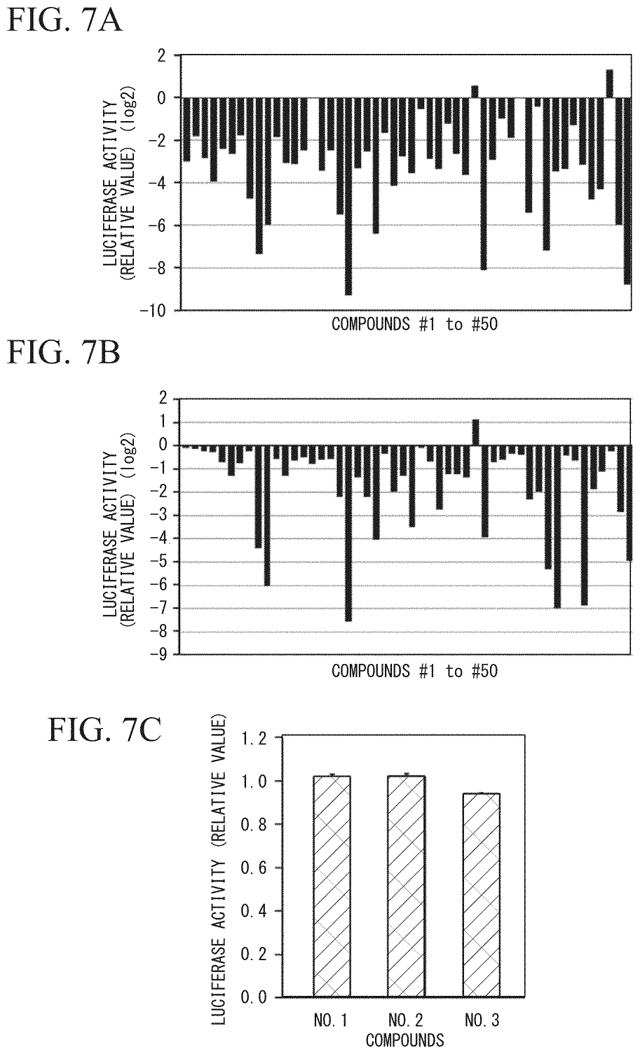

To get closer to the environment in the liver, a cell stably expressing an expression cassette in which a luciferase gene was ligated to the downstream side of the G0S2 promoter (−2030→+10) was constructed in the Hepa1-6 cell, which is a mouse hepatoma-derived cell. Using this cell, an effect of the candidate compounds on the luciferase activity was evaluated in the same manner as in the primary screening described above. FIG. 7A shows the results of the screening.

Further, in order to investigate whether the transcription inhibitory effect of the hit compounds has selectivity for the G0S2 promoter, studies were conducted focusing on the clock gene group involved in the expression of numerous genes in vivo and related to the maintenance of in vivo homeostasis.

The research of the present inventors suggests that the clock gene is also involved in the expression of the nuclear receptor RARα which controls the transcription of the G0S2 gene. Therefore, in order to exclude compounds having an effect on clock genes in normal cells, studies were conducted using the MEF cell line stably expressing an expression cassette in which a luciferase gene was ligated to the downstream side of the Bmal1 promoter. The results are shown in FIG. 7B.

As a result, it was suggested that numerous compounds could significantly inhibit the promoter activity of Bmal1. Therefore, as a result of extracting compounds which lead to no significant change in Bmal1 promoter activity and lower the activity of the G0S2 promoter (−2030→+10) to less than ½, three compounds of Compound No. 1, Compound No. 2 and Compound No. 3 remained as candidates. Compound No. 1, Compound No. 2 (hereinafter, sometimes referred to as “NS-3-008”) and Compound No. 3 are respectively represented by General Formulas (12), (3), and (13).

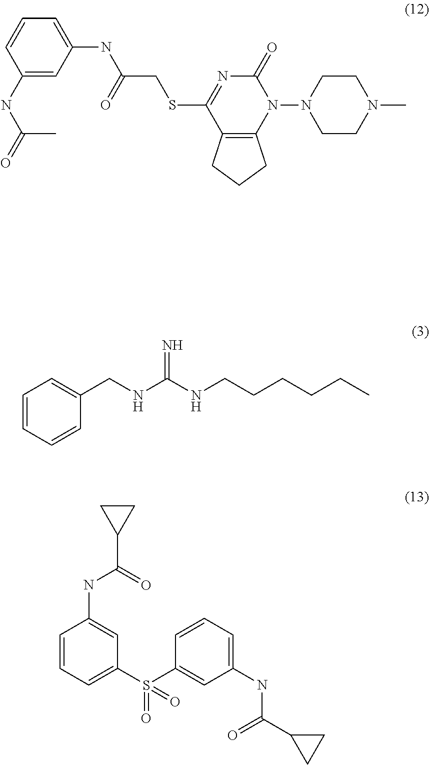

Therefore, further, using an MEF cell line stably expressing an expression cassette in which a luciferase gene was ligated to the downstream side of the Per2 promoter, the drug efficacy evaluation of these three compounds was carried out. As a result, all three of the remaining hit compounds had no effect on the Per2 promoter activity. The results are shown in FIG. 7C.

### Experimental Example 7

(Effect of Hit Compounds on Endogenous mRNA Expression)

The effect of the compound on the expression level of mRNA of the G0S2 gene was evaluated for the three compounds extracted so far. 24 hours after seeding with Hepa1-6 cells, the cells were exposed to hit compounds at 10 μM and sampling and total RNA extraction were carried out 24 hours later. Subsequently, changes in the expression level of mRNA of the G0S2 gene were measured by a qRT-PCR method.

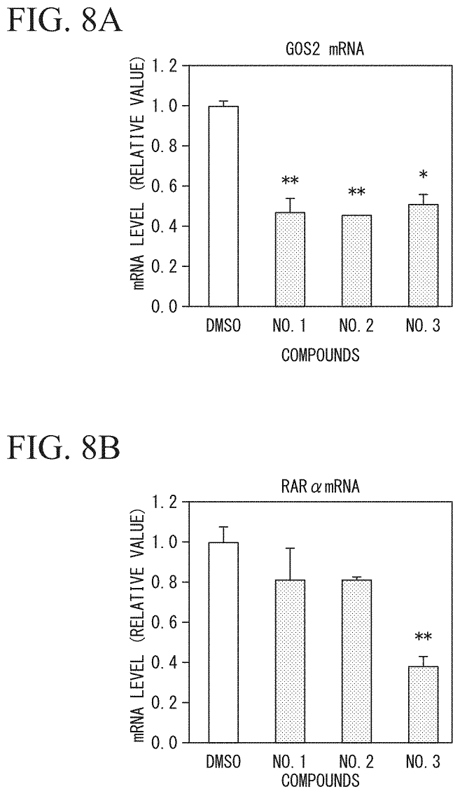

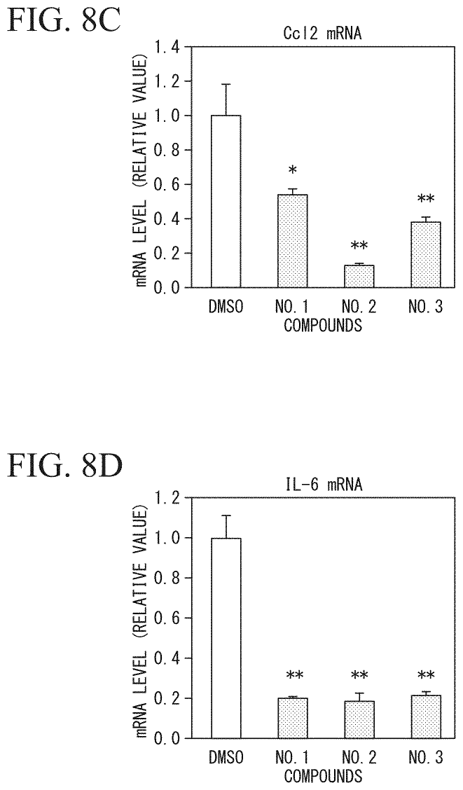

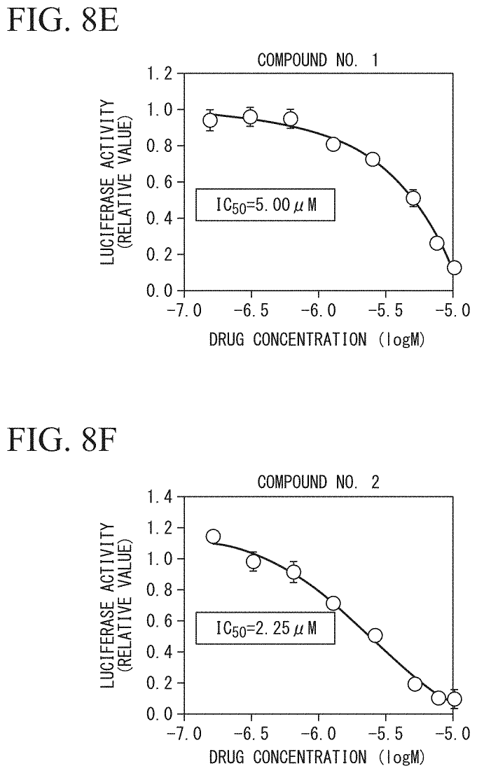

As a result, as shown in FIG. 8A, it was revealed that the expression level of mRNA of the G0S2 gene was decreased to about half in the case of all the compounds. Furthermore, as shown in FIG. 8B, there was no change in the expression level of the nuclear receptor RARα, which is known to be involved in the control of the transcription of G0S2, in the case of Compound No. 1 and Compound No. 2, but there was a significant decrease in the expression level of the nuclear receptor RARα in the case of Compound No. 3. Also, as shown in FIGS. 8C and 8D, there was a significant decrease in expression levels of mRNAs of Ccl2 and interleukin (IL)-6, which are downstream factors of an NF-κB signal for which G0S2 contributes as a regulator, in the case of exposure to any of the compounds. Based on these results, Compound No. 3 was excluded from the candidates, and Compound No. 1 and Compound No. 2 were taken as candidate compounds.

### Experimental Example 8

(Calculation of IC₅₀ of Hit Compounds)

For the evaluation of drug efficacy of Compound Nos. 1 and 2, which are hit compounds, an IC₅₀ was calculated. The O-4000E cell line was seeded, and Compound Nos. 1 and 2 were serially diluted in eight steps so as to have final concentrations of 0.15625 μM to 10 μM, followed by exposure of the cells to the compounds. The luciferase activity was measured 24 hours after the exposure, and the curve fitting was carried out to calculate an IC₅₀ of the compounds. As a result, as shown in FIG. 8E, the IC₅₀ of Compound No. 1 was calculated to be 5.00 μM, and as shown in FIG. 8F, the IC₅₀ of Compound No. 2 was calculated to be 2.25 μM.

### Experimental Example 9

(Evaluation of Drug Efficacy of Compound No. 2 on Normal Mice)

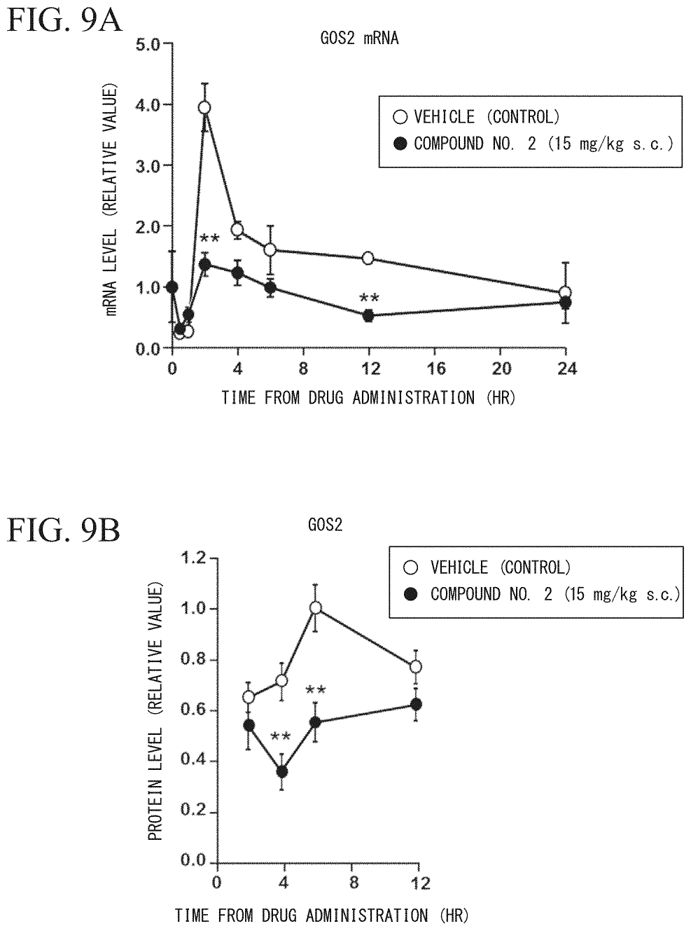

An effect of Compound No. 2 with a lower IC₅₀ value on the expression level of G0S2 mRNA in the liver of normal mice was examined. Compound No. 2 was subcutaneously administered on the dorsal region of ICR male mice at a dose of 15 mg/kg. The expression level of G0S2 mRNA in the liver at 30 minutes, 1, 2, 4, 6, 12, and 24 hours after the administration of Compound No. 2 was measured. As a result, as shown in FIG. 9A, it was found that the expression level of G0S2 mRNA tended to decrease to about half at 4 hours after administration, and it was found that the expression level of G0S2 mRNA was significantly decreased at 2 hours and 12 hours. Furthermore, as shown in FIG. 9B, when the expression level of the G0S2 protein in the liver from 2 hours to 12 hours after administration was measured, a significant decrease in the protein expression level was observed in the Compound No. 2-administered group at 4 hours and at 6 hours after administration.

### Experimental Example 10

(Evaluation 1 of Antagonistic Effect of Compound No. 2 on Inflammatory Signal)

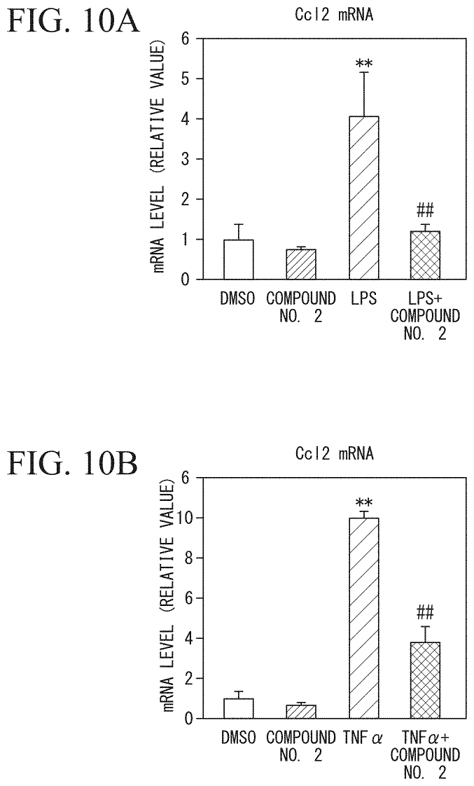

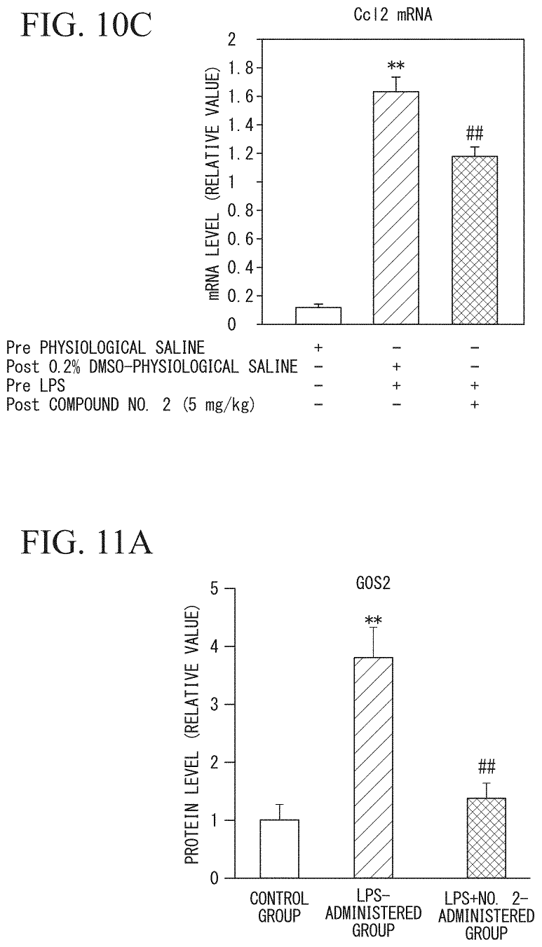

Next, whether or not Compound No. 2 whose effect in vivo was confirmed has an effect to antagonize the activation of an inflammatory signal was examined. Hepa1-6 cells were exposed to Compound No. 2 at a dose of 10 μM simultaneously with exposure to lipopolysaccharide (LPS) or TNFα which activates NF-κB for which the G0S2 protein acts as a regulator. As shown in FIGS. 10A and 10B, the cells were recovered 12 hours after the exposure and the expression level of Ccl2 mRNA was measured. As a result, the expression of Ccl2 mRNA increased by the exposure to LPS or TNFα was significantly inhibited by the exposure to Compound No. 2. In addition, 25 μg of LPS was administered to the tail vein of a mouse to thereby construct a hepatitis model. 30 minutes after the administration of LPS, Compound No. 2 (5 mg/kg) was administered to the tail vein of the mouse, and the liver was sampled at 6 hours. As a result, as shown in FIG. 10C, the expression level of Ccl2 mRNA in the liver was significantly increased as compared to the control group (physiological saline-administered group), but the expression of Ccl2 mRNA was significantly inhibited in the Compound No. 2-administered group.

### Experimental Example 11

(Evaluation 2 of Antagonistic Effect of Compound No. 2 on Inflammatory Signal)

Whether or not Compound No. 2 has an effect to antagonize the activation of an inflammatory signal in vivo was examined under the conditions different from those of Experimental Example 10.

LPS (10 μg/mouse) was administered to ICR male mice by tail vein injection to prepare an LPS-administered group. In addition, a mouse to which physiological saline was administered instead of LPS was prepared and used as a control group. Subsequently, the LPS-administered group was divided into two groups, in which 1 hour after the administration of LPS, Compound No. 2 was subcutaneously administered at a dose of 5 mg/kg to the dorsal region of mice of one group which was then served as an LPS+No. 2-administered group. Subsequently, 5 hours after the administration of Compound No. 2, mice of each group were sacrificed and the liver was harvested.

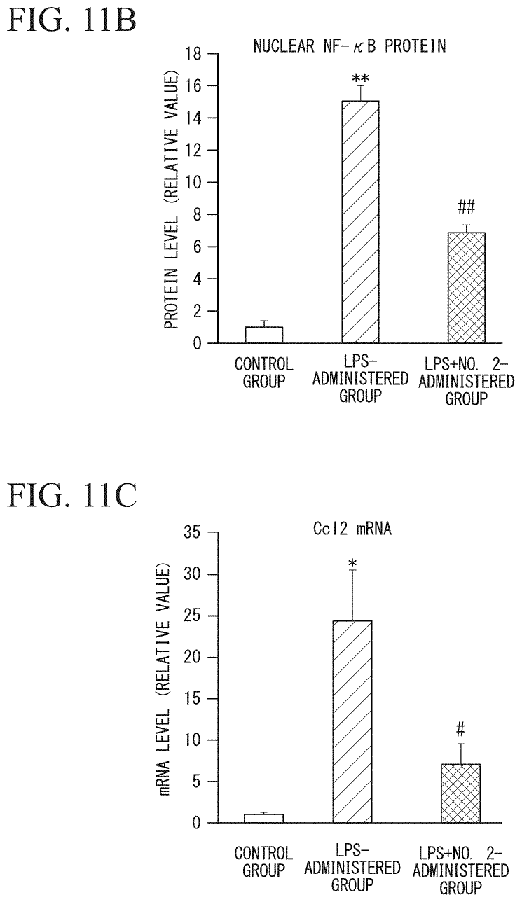

Subsequently, the expression level of the G0S2 protein in the liver of mice of each group obtained was quantified by Western blotting. FIG. 11A is a graph showing the quantitative results of the G0S2 protein.

As a result, the expression of G0S2 was significantly increased in the LPS-administered group as compared to the control group. Furthermore, the expression of G0S2 was significantly inhibited in the LPS+No. 2-administered group as compared to the LPS-administered group.

For the liver of mice of each group, the nuclear abundance of the p65 protein, which is one of the constituent proteins of NF-κB, was quantified by Western blotting. FIG. 11B is a graph showing the quantitative results of the p65 protein.

As a result, the nuclear abundance of the p65 protein was significantly increased in the LPS-administered group as compared to the control group. Furthermore, the nuclear abundance of the p65 protein was significantly decreased in the LPS+No. 2-administered group as compared to the LPS-administered group.

In addition, for the liver of mice of each group, the expression level of mRNA of Ccl2, which is a target gene of NF-κB, was quantified by real-time PCR. FIG. 11C is a graph showing the quantitative results of the expression level of mRNA of Ccl2.

As a result, the expression level of Ccl2 mRNA was significantly increased in the LPS-administered group as compared to the control group. Furthermore, the expression level of Ccl2 mRNA was significantly decreased in the LPS+No. 2-administered group as compared to the LPS-administered group.

The above results further support that Compound No. 2 antagonizes an inflammatory signal in vivo and has an effect of inhibiting the elevated expression of chemokines via NF-κB.

### Experimental Example 12

(Evaluation of Drug Efficacy of Compound No. 2 on Neuropathic Pain)

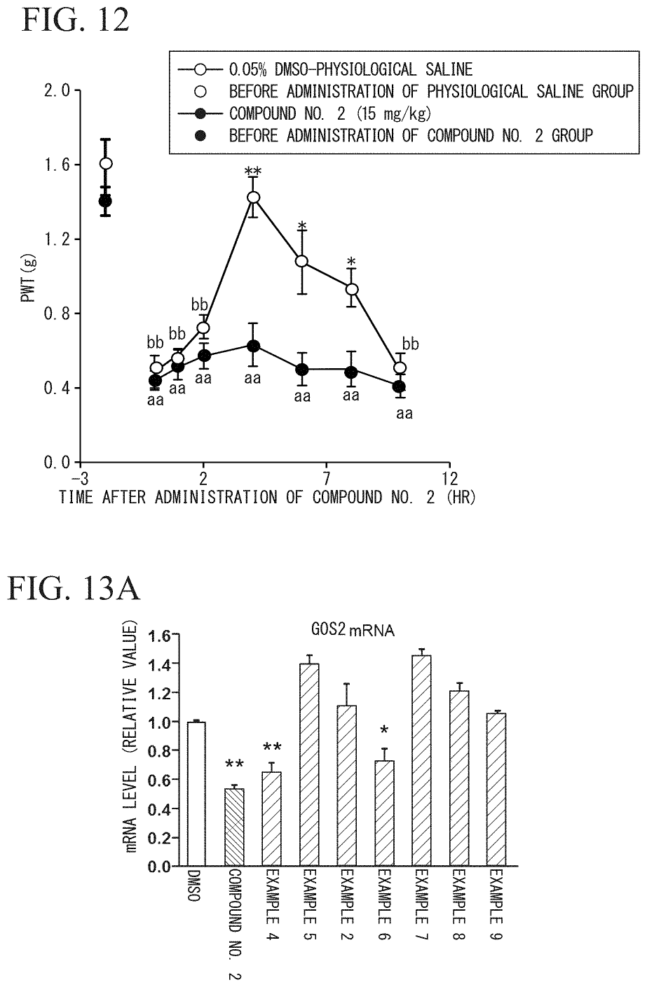

Whether or not Compound No. 2 which was found to be effective in an inflammation model has an effect of antagonizing neuropathic pain was examined. The neuropathic pain model constructed by cancer transplantation activates NF-κB. Therefore, a dose of 15 mg/kg of Compound No. 2 was subcutaneously administered to a model mouse in which the Bonferroni test was carried out at 2 weeks after cancer transplantation and the pain intensity was measured to confirm that pain was induced. As shown in FIG. 12, as a result of measuring the pain intensity over time after administration, pain was significantly relieved at 4, 6 and 8 hours in the Compound No. 2-administered group. Further, similar effects were also observed in a sciatic nerve partial damage model mouse.

### Experimental Example 13

(Synthesis of Compounds)

Compounds of Example 1 and Example 2 were synthesized according to the following scheme.

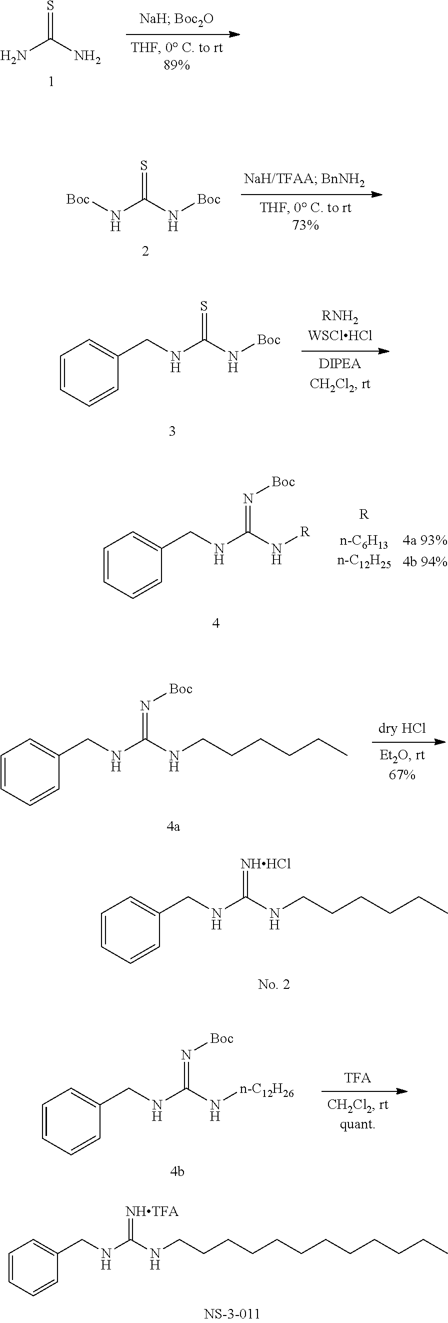

First, Compound 2 was obtained by reacting thiourea (Compound 1) with di-tert-butyl dicarbonate in the presence of sodium hydride. Subsequently, after converting Compound 2 into Compound 3 (see Yin B, et al., Tetrahedron Lett., 49, 3687, 2008), Compound 4a (see Poss M A, et al., Tetrahedron Lett., 33, 5933, 1992) was obtained in a yield of 93% by condensation of Compound 3 with hexylamine in the presence of a water-soluble carbodiimide, and Compound 4b was obtained in a yield of 94% by condensation of Compound 3 with dodecylamine. By reacting Compound 4a with a solution of hydrogen chloride ether, the compound of Example 1 (Compound No. 2) was obtained in a yield of 67%.

(Compound No. 2)

¹H NMR (500 MHz, DMSO-d₆) δ8.10 (1H, bs), 7.79 (1H, bs), 7.56 (2H, bs), 7.41-7.27 (5H, m), 4.43 (2H, d, J=6.2 Hz), 3.15 (2H, q, J=6.8 Hz), 1.51-1.41 (2H, m), 1.32-1.21 (6H, m), 0.87 (3H, t, J=6.7 Hz).

Further, a compound of Example 2 (NS-3-011) was quantitatively obtained by reacting Compound 4b with trifluoroacetic acid in dichloromethane.

(NS-3-011)

¹H NMR (500 MHz, DMSO-d₆) δ7.99 (1H, bs), 7.66 (1H, t, J=5.2 Hz), 7.50 (2H, bs), 7.42-7.35 (2H, m), 7.33-6.26 (3H, m), 4.41 (2H, d, J=6.1 Hz), 3.14 (2H, q, J=6.8 Hz), 1.53-1.41 (3H, m), 1.32-1.16 (17H, m), 0.86 (3H, t, J=6.8 Hz).

Further, a compound of Example 3 (NS-3-054) represented by General Formula (4) was also synthesized.

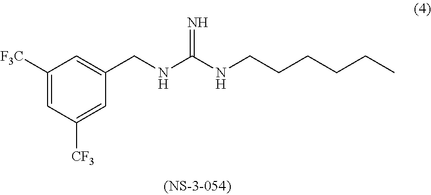

¹H NMR (500 MHz, DMSO-d₆) δ8.21 (1H, t, J=6.4 Hz), 8.07 (1H, s), 8.01 (2H, s), 7.82 (1H, t, J=5.6 Hz), 7.61 (2H, bs), 4.61 (2H, d, J=6.4 Hz), 3.15 (2H, q, J=6.8 Hz), 1.50-1.38 (2H, m), 1.29-1.12 (6H, m), 0.84 (3H, t, J=6.8 Hz).

Further, a compound of Example 4 (NS-3-005) represented by General Formula (5) was also synthesized.

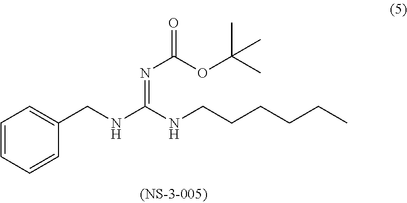

¹H NMR (500 MHz, CDCl₃) δ7.38-7.28 (5H, m), 4.48 (2H, bs), 3.15 (2H, bs), 1.62-1.48 (11H, m), 1.35-1.16 (6H, m), 0.86 (3H, t, J=6.5 Hz).

Further, a compound of Example 5 (NS-3-010) represented by General Formula (6) was also synthesized.

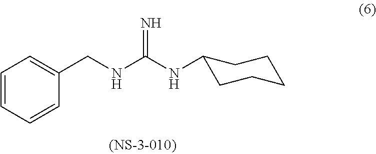

¹H NMR (500 MHz, DMSO-d₆) δ7.77 (1H, bs), 7.45 (2H, bs), 7.42-7.37 (3H, m), 7.34-7.28 (3H, m), 4.41 (2H, d, J=6.1 Hz), 3.47-3.37 (1H, m), 1.86-1.77 (2H, m), 1.73-1.65 (2H, m), 1.61-1.53 (1H, m), 1.35-1.08 (5H, m).

Further, a compound of Example 6 (NS-3-013) represented by General Formula (7) was also synthesized.

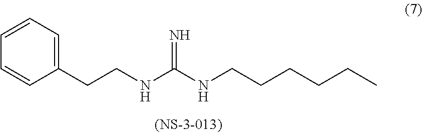

¹HNMR (500 MHz, DMSO-d₆) δ7.50-7.41 (2H, m), 7.37 (2H, bs), 7.34-7.29 (2H, m), 7.28-7.21 (3H, m), 3.43-3.35 (2H, m), 3.09 (2H, q, J=7.6 Hz), 2.80 (2H, t, J=6.8 Hz), 1.48-1.38 (2H, m), 1.34-1.20 (6H, m), 0.88 (3H, t, J=6.8 Hz).

Further, a compound of Example 7 (NS-3-014) represented by General Formula (8) was also synthesized.

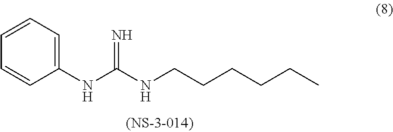

¹HNMR (500 MHz, DMSO-d₆) δ9.66 (1H, bs), 8.03-7.90 (1H, m), 7.71 (2H, bs), 7.48-7.41 (2H, m), 7.28 (1H, t, J=7.4 Hz), 7.22 (2H, d, J=7.7 Hz), 3.22 (2H, q, J=6.8 Hz), 1.59-1.48 (2H, m), 1.39-1.22 (6H, m), 0.89 (3H, t, J=6.8 Hz).

Further, a compound of Example 8 (NS-3-015) represented by General Formula (9) was also synthesized.

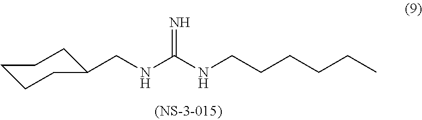

¹HNMR (500 MHz, DMSO-d₆) δ7.41-7.34 (2H, m), 7.31 (2H, bs), 3.11 (2H, q, J=6.9 Hz), 2.97 (2H, t, J=6.4 Hz), 1.76-1.58 (5H, m), 1.54-1.41 (3H, m), 1.36-1.07 (9H, m), 0.96-0.82 (5H, m).

Further, a compound of Example 9 (NS-3-016) represented by General Formula (10) was also synthesized.

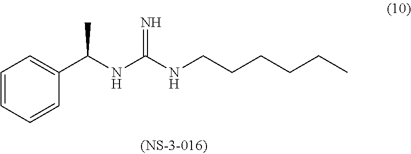

¹HNMR (500 MHz, DMSO-d6) δ7.94-7.84 (1H, m), 7.48 7.35 (5H, m), 7.35-7.27 (3H, m), 4.87-4.78 (1H, m), 3.18-3.03 (2H, m), 1.50-1.34 (5H, m), 1.31-1.13 (6H, m), 0.85 (3H, t, J=6.8 Hz).

Further, a compound of Example 10 (NS-3-086) represented by General Formula (11) was also synthesized.

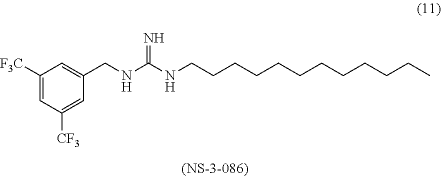

¹HNMR (500 MHz, DMSO-d₆) δ8.09 (1H, bs), 8.07 (1H, s), 8.00 (2H, s), 7.73 (1H, t, J=5.2 Hz), 7.56 (2H, bs), 4.60 (2H, d, J=6.4 Hz), 3.14 (2H, q, J=6.7 Hz), 1.48-1.38 (2H, m), 1.32-1.10 (18H, m), 0.86 (3H, t, J=6.8 Hz).

Further, a compound of Comparative Example 1 (NS-3-060) represented by General Formula (12) was also synthesized.

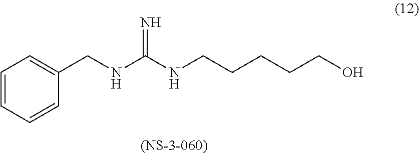

¹HNMR (500 MHz, DMSO-d₆) δ7.83 (1H, bs), 7.52 (1H, bs), 7.45 (2H, bs), 7.41-7.36 (2H, m), 7.34 7.27 (3H, m), 4.40 (2H, d, J=6.1 Hz), 4.38 (1H, t, J=6.5 Hz), 3.39 (2H, t, J=6.4 Hz), 3.19-3.10 (2H, m), 1.75-1.66 (1H, m), 1.55-1.37 (3H, m), 1.36-1.23 (2H, m).

### Experimental Example 14

(Discovery of Derivatives of Compound No. 2 and Evaluation of Drug Efficacy Thereof)

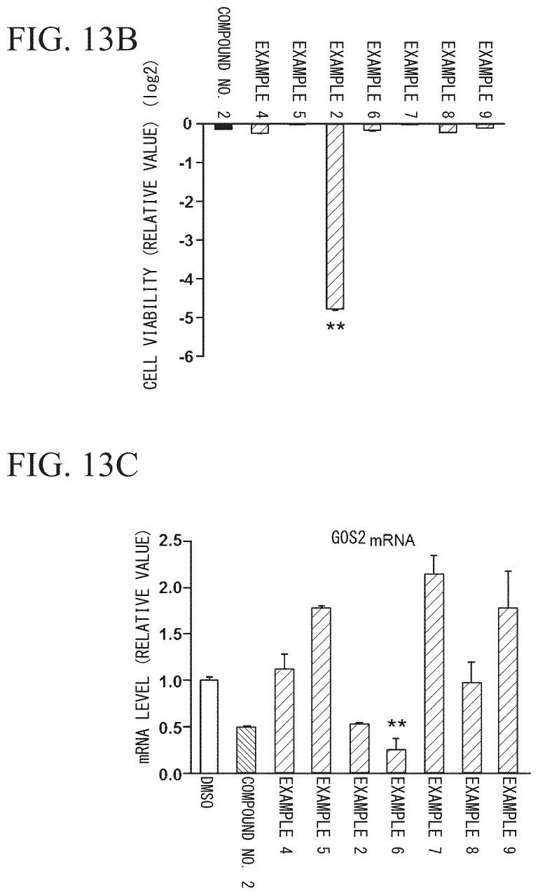

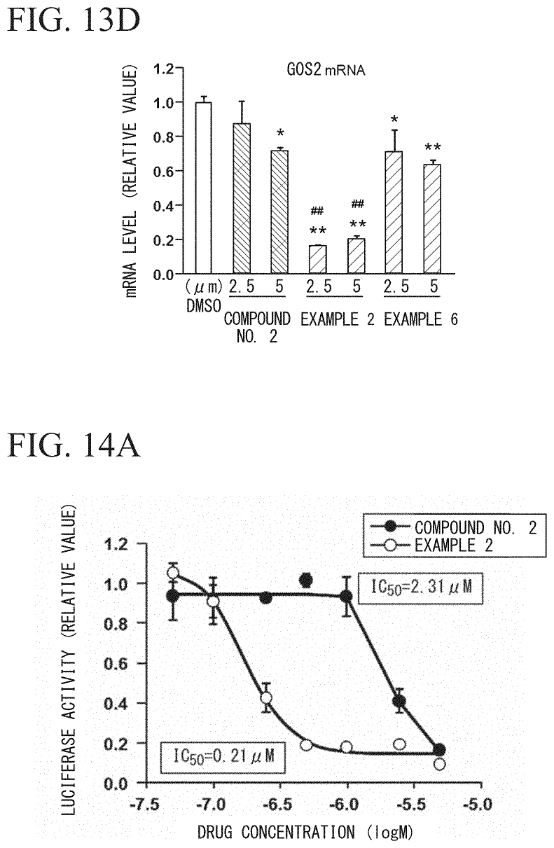

Hepa1-6 cells were exposed to 10 μM of each of seven derivatives of Compound No. 2 (Examples 2 and 4 to 9) and an effect of the derivative compounds on the expression level of G0S2 mRNA after 24 hours was evaluated. As a result, as shown in FIG. 13A, an effect of lowering the expression level of G0S2 mRNA up to the same extent as that of Compound 2 was recognized in the compounds of Example 4 (NS-3-005) and Example 6 (NS-3-013).

In addition, as shown in FIG. 13B, when these compounds were evaluated for their effect on cell viability by an ATP assay, a significant decrease in cell viability was observed only in the compound of Example 2 (NS-3-011).

Further, as shown in FIG. 13C, when the drug efficacy on human HepG2 cells was evaluated at an mRNA level, a significant decrease was observed in the compound of Example 6 (NS-3-013), and the same effect as that of Compound No. 2 was confirmed in the compound of Example 2 (NS-3-011).

Therefore, for the compound of Example 6 (NS-3-013) whose drug efficacy was observed in common in mouse hepatoma-derived cells, Hepa1-6 cells and human hepatoma-derived cells, HepG 2 cells and the compound of Example 2 (NS-3-011) by which cell death was observed at a concentration of 10 μM, the drug efficacy at a concentration of less than 10 μM was evaluated.

As a result, as shown in FIG. 13D, when Hepa1-6 cells were exposed to Compound No. 2, the compound of Example 2 (NS-3-011), and the compound of Example 6 (NS-3-013) at a concentration of 2.5 μM and 5 μM for 48 hours, Compound No. 2 exhibited a significant decrease in G0S2 mRNA expression level at the exposure to a concentration of 5 μM, and the compound of Example 2 (NS-3-011) and the compound of Example 6 (NS-3-013) exhibited a significant decrease in G0S2 mRNA expression level at the exposure to a concentration of both 5 μM and 2.5 μM. In addition, the compound of Example 2 (NS-3-011) exhibited a significant decrease in G0S2 mRNA expression level at any concentration, as compared to the group exposed to 5 μM of Compound No. 2.

### Experimental Example 15

(Comparison of Drug Efficacy Between Compound No. 2 and Compound of Example 2)

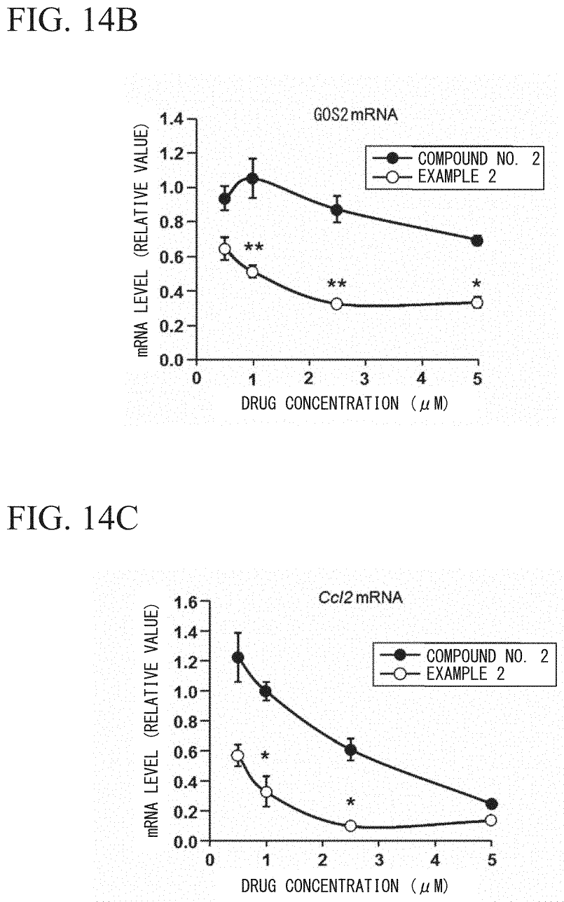

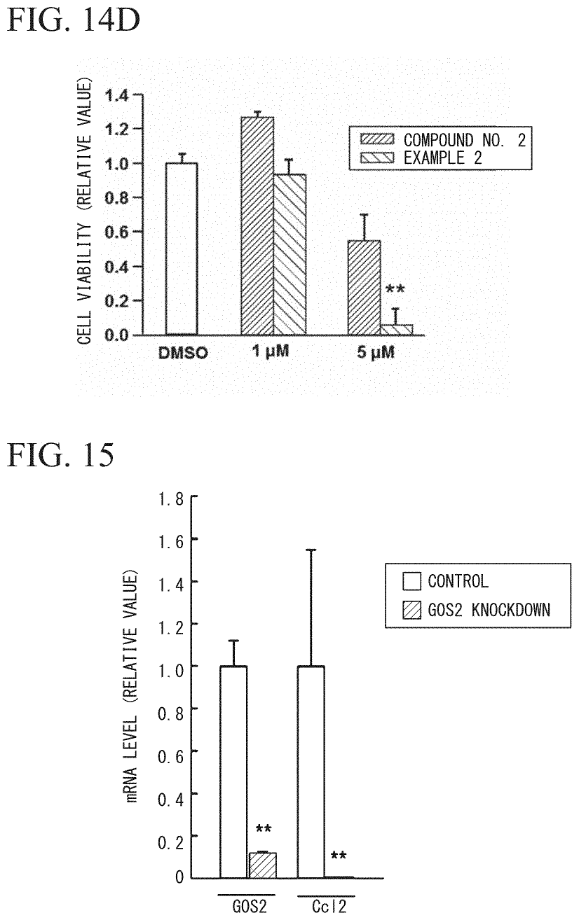

Detailed drug efficacy evaluation at low concentrations was carried out for the compound of Example 2 (NS-3-011) whose drug efficacy at a concentration of less than 10 μM was observed based on the studies so far and which is expected to have an effect equal to or more than that of Compound No. 2. First, using O-4000E cells, IC₅₀ of Compound No. 2 and the compound of Example 2 (NS-3-011) on the G0S2 promoter activity was calculated. As a result, as shown in FIG. 14A, Compound No. 2 exhibited an IC₅₀ of 2.31 μM, whereas the compound of Example 2 (NS-3-011) exhibited an IC₅₀ of 0.21 μM, which was found to exhibit equivalent drug efficacy in an amount of about 1/10.

Therefore, Hepa1-6 cells were exposed to Compound No. 2 and the compound of Example 2 (NS-3-011) in the range of 0.5 to 5 μM and an effect of the compounds on the expression level of endogenous G0S2 mRNA after 48 hours was evaluated. As a result, as shown in FIG. 14B, the compound of Example 2 (NS-3-011) exhibited a significantly higher effect in the range of 1 to 5 μM than Compound No. 2, and had an IC₅₀ of about 1 μM.

Similarly, when the expression level of Ccl2 mRNA was evaluated, as shown in FIG. 14C, the compound of Example 2 (NS-3-011) exhibited a significantly higher effect in the range of 1 to 2.5 μM than Compound No. 2. Moreover, as shown in FIG. 14D, the cell viability at this time was evaluated by an ATP assay, and as a result, there was a significant decrease in cell viability at the time of exposure to 5 μM of the compound of Example 2 (NS-3-011). However, it was suggested that the compound of Example 2 (NS-3-011) is a more useful compound than Compound No. 2 since no decrease in cell viability was observed at a concentration of 1 μM of the compound of Example 2 (NS-3-011), and the compound (NS-3-011) of Example 2 exhibited a stronger effect at 1 μM than Compound No. 2.

### Experimental Example 16

(Knockdown of G0S2 by miRNA)

DNA fragments having the base sequences set forth in SEQ ID NOs: 5 and 6 were synthesized and hybridized to form a double-stranded DNA fragment. Subsequently, the double-stranded DNA fragment thus formed was ligated to an expression vector for miRNA (trade name “BLOCK-iT™ Pol II miR RNAi Expression Vector Kits”, manufactured by Invitrogen). Subsequently, the constructed expression vector was transfected into NIH3T3 cells. 48 hours after transfection, expression levels of G0S2 and Ccl2 mRNAs were quantified by real-time PCR.

FIG. 15 is a graph showing the results of real-time PCR. As a result, it was revealed that the expression levels of G0S2 and Ccl2 mRNAs were decreased in the cells with knockdown of G0S2 as compared to the control cells.

### Experimental Example 17

(Evaluation of Drug Efficacy of Compounds)

10 μg of LPS was intraperitoneally administered to mice to construct a hepatitis model. 1 hour after the administration of LPS, the compound of Example 2 (NS-3-011), the compound of Example 3 (NS-3-054) and the compound of Example 10 (NS-3-086) were subcutaneously administered to the dorsal region of animals. 5 hours after the administration of each compound, the liver was harvested from each mouse and the expression level of Ccl2 was quantified by real-time PCR.

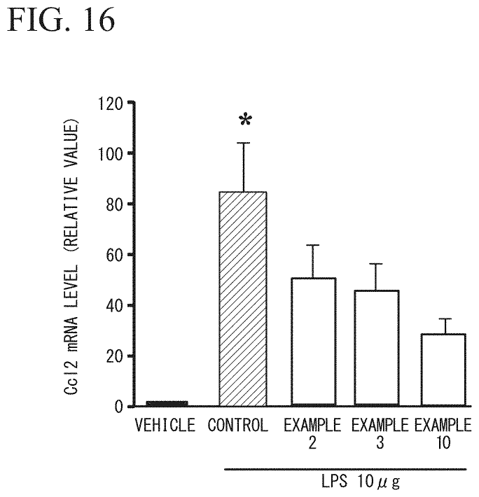

FIG. 16 is a graph showing the results of real-time PCR. In the figure, the “VEHICLE” represents mice that did not receive LPS and the “CONTROL” represents mice that received physiological saline in place of the compound.

As a result, it was revealed that the expression level of Ccl2 mRNA was decreased in the mice of the compound-administered group as compared to the mice of the control group. A decrease in the expression level of Ccl2 mRNA was observed with any of the compounds.

### Experimental Example 18

(Identification of Target Protein of Compound of Example 2)

Mouse hepatocytes were homogenized to extract proteins from the cytoplasm and nucleus. Then, a compound (#142) obtained by PEG biotinylation of the compound of Example 2 (NS-3-011) inhibiting the expression of G0S2, or a compound (#151) obtained by PEG biotinylation of the compound of Comparative Example 1 (NS-3-060) not inhibiting the expression of G0S2 was mixed with the above-obtained protein, followed by incubation. The chemical formulae of Compounds #142 and #151 are shown in General Formulae (13) and (14), respectively.

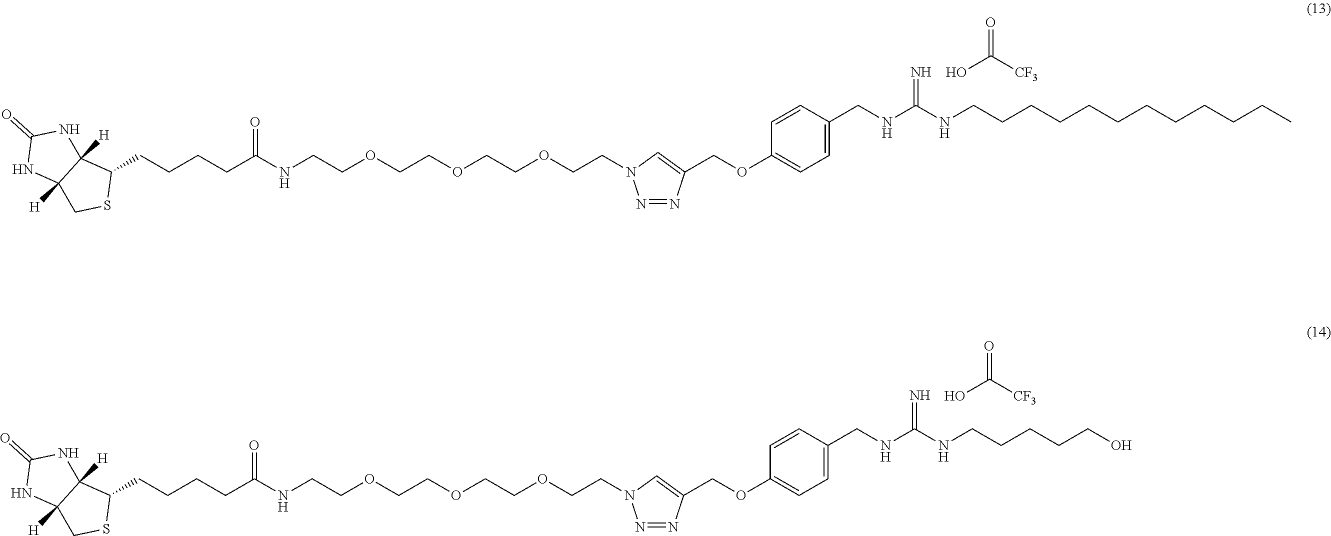

Thereafter, a pull-down assay was carried out with avidin-labeled magnetic beads (hereinafter, sometimes referred to as “avidin beads”), and the compounds and proteins bound to the avidin beads were subjected to acrylamide gel electrophoresis. The acrylamide gel after subjecting to electrophoresis was negatively stained, a band of a protein specifically bound to Compound #142 was excised, and the protein was identified by an LC/MS/MS analysis.

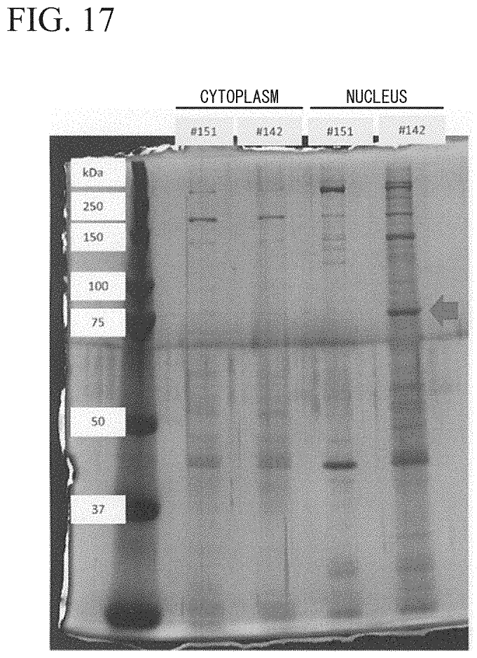

FIG. 17 is a photograph showing the results of acrylamide gel electrophoresis. Analysis of the band of the protein of about 75 kDa indicated by the arrow in the figure revealed that the protein is Hsd17b4 (Peroxisomal multifunctional enzyme type 2, Uniprot accession number: P51660).

### Experimental Example 19

(Evaluation of Drug Efficacy of Compound of Example 1 Using Non-Alcoholic Steatohepatitis Model Mouse)

In order to confirm an effect of the compound on the initial inflammation of a non-alcoholic steatohepatitis (NASH) model, mice were fed with a diet for constructing the NASH model (Product # “A06071302”, Research Diets Inc.) for 1 week. The compound of Example 1 (2.5, 5.0 mg/kg) was orally administered to animals at 19 o'clock every day from the start of feeding. As a control, mice fed with a normal diet and orally administered with the same dose of 0.5% DMSO-added water as that of the compound were used.

The liver was harvested one week after the start of feeding, and expression levels of Ccl2, αSMA, collagen type I α2, and collagen type I al genes were measured by real-time PCR. Note that αSMA, collagen type I α2, and collagen type I al are markers that are indicators of hepatic fibrosis.

In addition, blood was collected one week after the start of feeding, and the blood drug concentration at 2 hours after the drug administration was measured by LC/MS/MS.

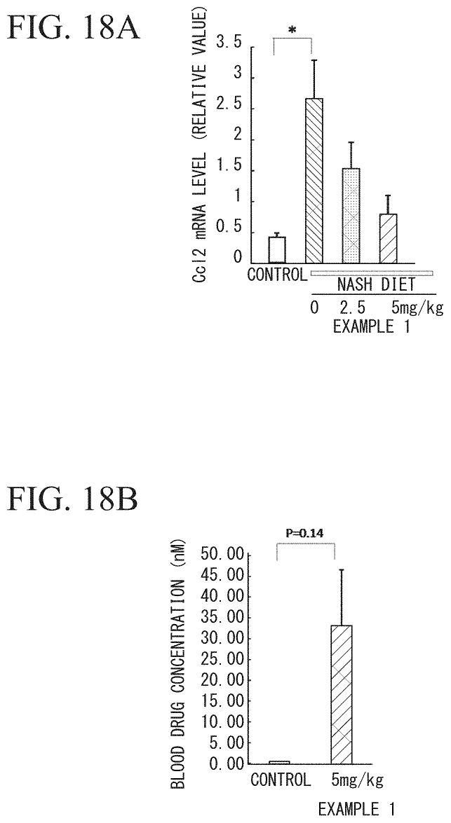

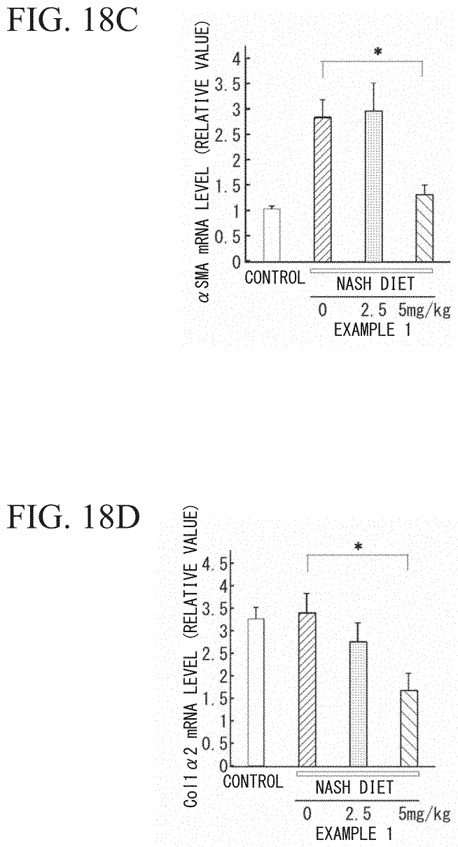

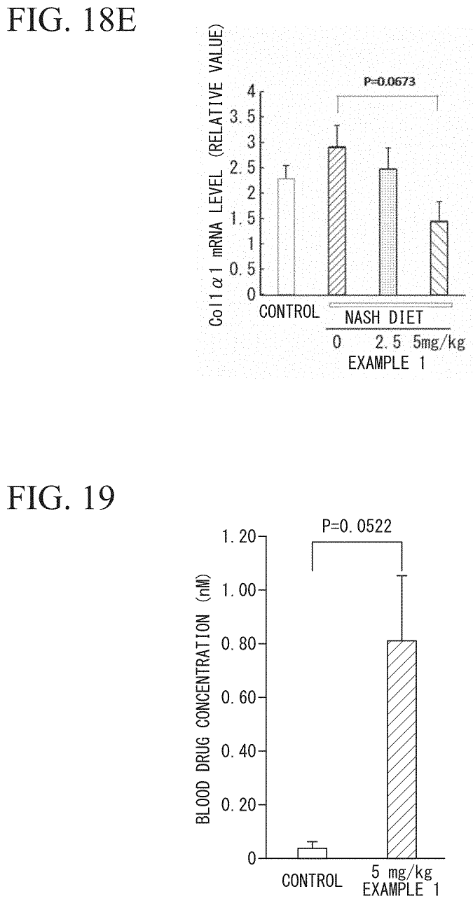

FIG. 18A is a graph showing the expression level of Ccl2 in the liver of a NASH model mouse. As shown in FIG. 18A, it was revealed that the expression level of Ccl2 is decreased by administration of the compound.

FIG. 18B is a graph showing the blood drug concentration of the compound of Example 1. As shown in FIG. 18B, the mean blood concentration of the compound of Example 1 was 32 nM.

FIGS. 18C, 18D, and 18E are graphs showing the results of quantifying the expression levels of αSMA, collagen type I α2 (Col1α2), and collagen type I al (Col1α1) genes, respectively. As shown in FIGS. 18C, 18D, and 18E, it was revealed that the expression levels of αSMA, collagen type I α2, and collagen type I al genes are significantly decreased in the group to which 5 mg/kg of the compound of Example 1 was administered.

### Experimental Example 20

(Daily Administration Test of Compound of Example 1)

The compound of Example 1 (5 mg/kg) was orally administered to mice at 19 o'clock every day. Blood was collected 21 days after the start of administration of the compound and the blood concentration of the compound 24 hours after the drug administration was measured by LC/MS/MS. As a control, mice fed with a normal diet and orally administered with the same dose of 0.5% DMSO-added water as that of the compound were used.

FIG. 19 is a graph showing the blood drug concentration of the compound of Example 1. As shown in FIG. 19, the mean blood concentration of the compound of Example 1 was 0.8 nM.

### Experimental Example 21

(Measurement of Blood Concentration of Compound of Example 1)

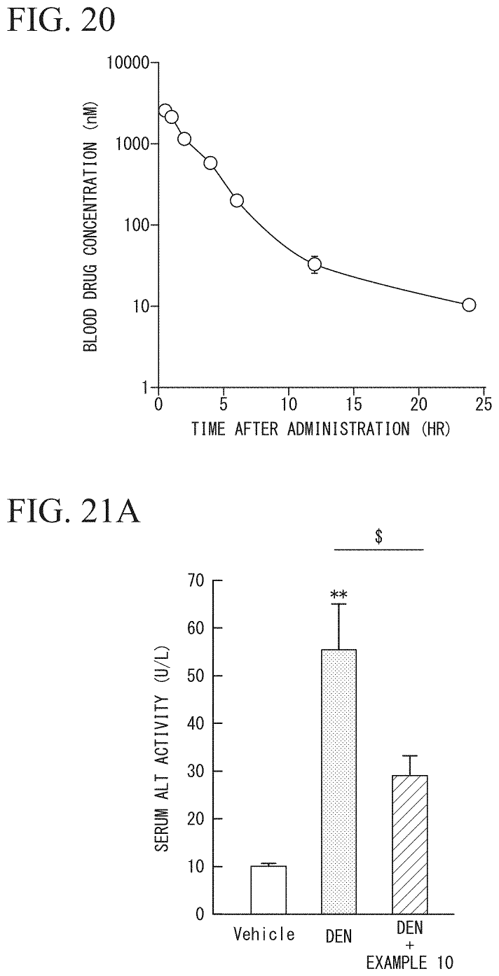

The compound of Example 1 (15 mg/kg) was subcutaneously administered to the dorsal region of ICR mice. Blood was collected at 0.5, 1, 2, 4, 6, 12 and 24 hours after the administration of the compound, and the blood concentration of the compound was measured by LC/MS/MS. FIG. 20 is a graph showing the blood drug concentration of the compound of Example 1.

### Experimental Example 22

(Examination of Effect of Compound on ALT Activity in Serum of DEN-Induced Hepatitis/Hepatoma Mouse Model)

Diethylnitrosamine (DEN) dissolved in water at a concentration of 80 mg/L as drinking water was administered in drinking water to ICR mice, thereby inducing chronic inflammation in the liver. In addition, a group in which 15 mg/kg of the compound of Example 10 (NS-3-086) was administered in drinking water from immediately after the start of administration of DEN in drinking water was prepared. As a control group, a group given tap water as drinking water was used.

Subsequently, 8 weeks and 12 weeks after the start of administration of DEN in drinking water, blood was collected from the tails of mice of each group, and the alanine aminotransferase (ALT) activity in serum was measured using a commercially available kit (“Transaminase CII-Test Wako”, manufactured by Wako Pure Chemical Industries, Ltd.).

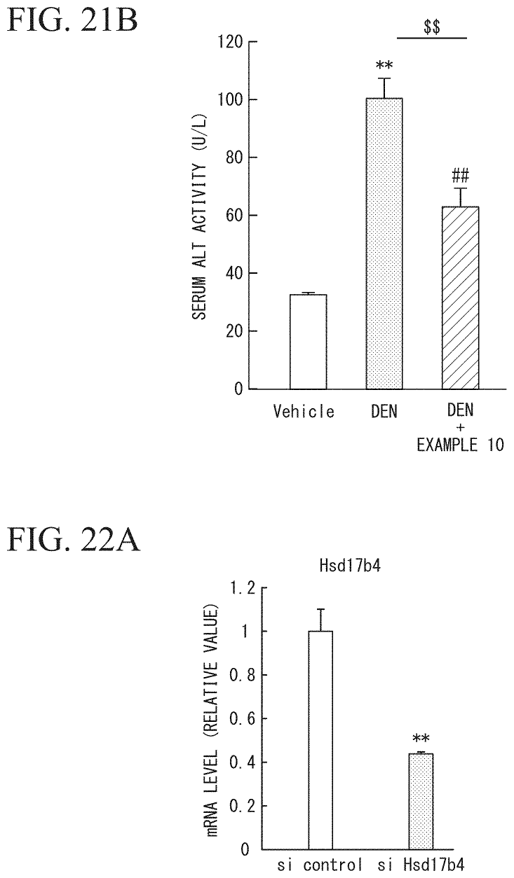

FIGS. 21A and 21B are graphs showing the serum ALT activity at 8 weeks (FIG. 21A) and 12 weeks (FIG. 21B) after the start of administration of DEN in drinking water. In FIGS. 21A and 21B, the “VEHICLE” shows the results of the control group, the “DEN” shows the results of the DEN-administered group, and the “DEN+EXAMPLE 10” shows the results of the group to which DEN and the compound of Example 10 were administered.

As a result, it was revealed that the ALT activity was significantly higher in the DEN drinking water group than in the control group. In addition, it was revealed that an increase in serum ALT activity by DEN drinking water was inhibited by co-administering the compound of Example 10.

The above results indicate that DEN-induced hepatic inflammation was inhibited by administration of the compound of Example 10.

### Experimental Example 23

(Examination of Effect of Hsd17b4 Knockdown on G0S2 mRNA Expression Level and Drug Efficacy of Compound)

Hepα1-6 cells, which are mouse hepatoma-derived cells, were divided into two groups, in which one group was transfected with siRNA against Hsd17b4 and the other group was transfected with control siRNA. The base sequence of the sense strand of siRNA against Hsd17b4 is set forth in SEQ ID NO: 7, and the base sequence of the antisense strand thereof is set forth in SEQ ID NO: 8. In addition, commercially available siRNA (Life Technologies, Inc., the base sequence of the antisense strand is set forth in SEQ ID NO: 4) was used as the control siRNA.

Subsequently, each cell was recovered after 48 hours and total RNA was extracted. Subsequently, the expression level of the Hsd17b4 gene was quantified by real-time PCR.

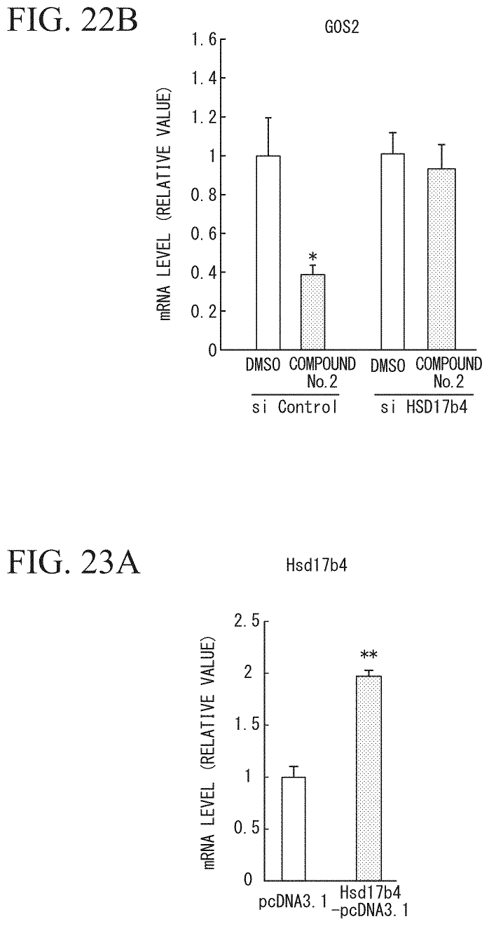

FIG. 22A is a graph showing the results of real-time PCR. As a result, it was confirmed that the expression of Hsd17b4 was inhibited by the transfection of the cells with siRNA against Hsd17b4.

Subsequently, Hepa1-6 cells were transfected with siRNA against Hsd17b4 or control siRNA in the presence (10 μM) or absence (indicated as “DMSO” in FIG. 22B) of Compound No. 2. Subsequently, each cell was recovered after 48 hours and total RNA was extracted. Subsequently, the expression level of the G0S2 gene was quantified by real-time PCR.

FIG. 22B is a graph showing the results of real-time PCR. As a result, it was revealed that the expression level of the G0S2 gene was significantly decreased in the presence of Compound No. 2. Furthermore, it was revealed that the effect of lowering the expression level of the G0S2 gene by Compound No. 2 disappeared by the transfection of the cells with siRNA against Hsd17b4.

Based on the above results, it was revealed that Compound No. 2 controls the expression of the G0S2 gene via the Hsd17b4 protein.

### Experimental Example 24

(Examination of Effect of Overexpression of Hsd17b4 Gene on G0S2 mRNA Expression Level)

An Hsd17b4 expression vector (Hsd17b4-pcDNA3.1) or an empty vector (pcDNA3.1) was transfected into Hepa1-6 cells, which are mouse hepatoma-derived cells.

Subsequently, each cell was recovered after 48 hours and total RNA was extracted. Subsequently, the expression level of the Hsd17b4 gene or the G0S2 gene was quantified by real-time PCR.

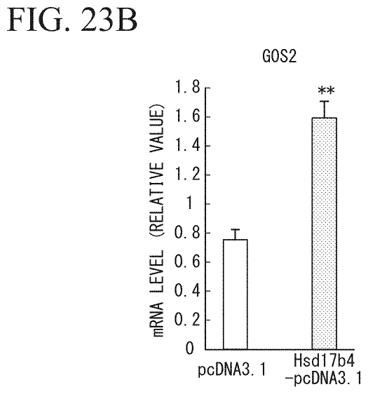

FIGS. 23A and 23B are graphs showing the results of real-time PCR. FIG. 23A shows the results of quantifying the expression level of the Hsd17b4 gene and FIG. 23B shows the result of quantifying the expression level of the G0S2 gene.

As a result, as shown in FIG. 23A, it was confirmed that the expression level of the Hsd17b4 gene was significantly increased by the transfection of the cells with the expression vector of Hsd17b4. In addition, as shown in FIG. 23B, it was revealed that the expression level of the G0S2 gene was significantly increased by the transfection of the cells with the expression vector of Hsd17b4.

Based on the above results, it was revealed that the expression level of the G0S2 gene is controlled via the Hsd17b4 protein.

### Experimental Example 25

(Examination of Effect of Compound on Transcriptional Activity Regulatory Region of G0S2)

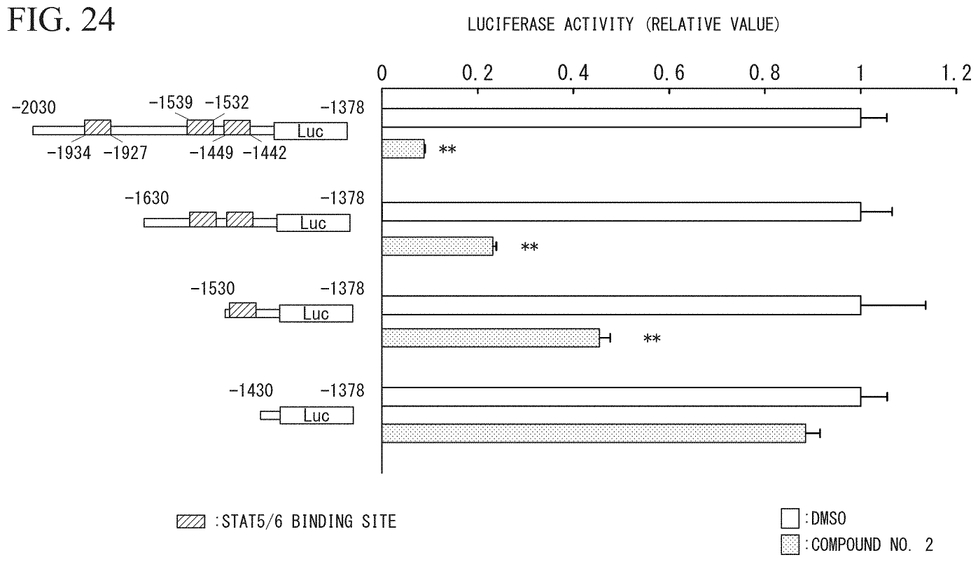

As shown in FIG. 24, expression vectors in which cDNA of luciferase was ligated to the downstream side of the G0S2 transcriptional activity regulatory region of each length were constructed. Subsequently, these expression vectors were transfected into NIH3T3 cells, which are mouse fibroblast cells, to construct cells stably expressing luciferase.

Subsequently, Compound No. 2 or DMSO (control) at a final concentration of 10 μM was added to the medium of each cell. Subsequently, the cells were recovered after 24 hours and the luciferase activity was measured.

FIG. 24 is a graph showing the results of measuring the luciferase activity of each cell. As a result, it was revealed that when the STAT5/6 binding region present in the G0S2 transcriptional activity regulatory region disappears, an effect of inhibiting the expression of luciferase by the addition of Compound No. 2 is decreased.

Based on the above results, it was considered that Compound No. 2 may act through the transcription of STAT5 or STATE.

### Experimental Example 26

(Examination on Effect of STAT5 on G0S2 Transcriptional Activity)

An expression vector in which cDNA of luciferase is ligated to the downstream side of a G0S2 transcriptional activity regulatory region and a STAT5 expression vector or an empty vector (pcDNA 3.1, control) were transfected into NIH3T3 cells, which are mouse fibroblasts. Subsequently, the cells were recovered after 24 hours and the luciferase activity was measured.

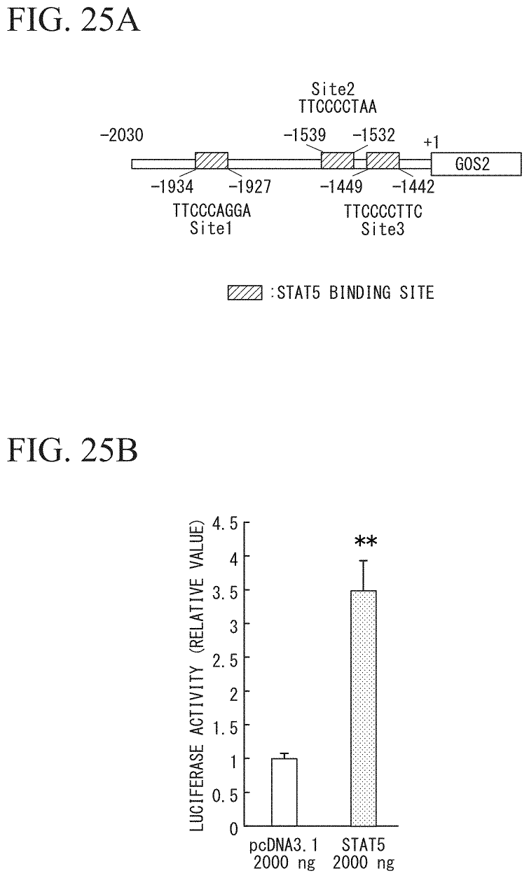

FIG. 25A is a diagram showing the structure of the transcriptional activity regulatory region of G0S2. FIG. 25B is a graph showing the results of measuring a luciferase activity. As a result, it was revealed that the cells transfected with the STAT5 expression vector exhibited a significantly higher luciferase activity than the control group.

Based on the above results, it was revealed that the transcriptional activity of the G0S2 gene is directly controlled by STAT5.

### Experimental Example 27

(Examination of Effect of Compound on Nuclear Abundance of STAT5 Protein)

Compound No. 2 or DMSO (control) at final concentrations of 2.5, 5 and 10 μM was added to the medium of NIH3T3 cells, which are mouse fibroblasts. Subsequently, the cells were recovered after 24 hours, the nuclear protein was prepared, and the abundance of the STAT5 protein was measured by Western blotting.

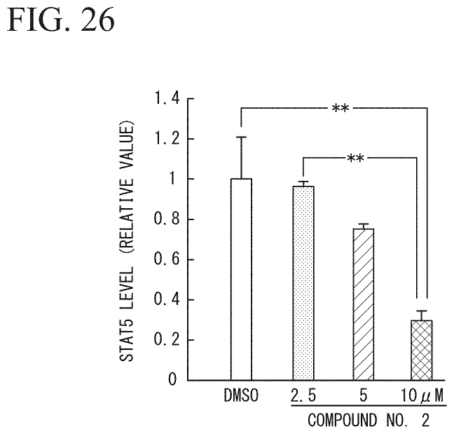

FIG. 26 is a graph showing the results of Western blotting. As a result, the nuclear abundance of the STAT5 protein was decreased in a dose-dependent manner of Compound No. 2 in the cells to which Compound No. 2 was added, in comparison with the control.

Based on the above results, it was revealed that Compound No. 2 controls the expression of the G0S2 gene by regulating the nuclear abundance of STAT5.

### Experimental Example 28

(Measurement of Blood and Liver Concentration of Compound No. 2)

Compound No. 2 (15 mg/kg) was orally administered to mice. Blood was collected at 0 minutes, 5 minutes, 15 minutes, 30 minutes, 1 hour, 2 hours, 4 hours, 6 hours and 12 hours after the administration of Compound No. 2 and the blood concentration of the compound was measured by LC/MS/MS. In addition, the liver was harvested at 0 minutes, 5 minutes, 15 minutes, 30 minutes, 1 hour and 6 hours after the administration of Compound No. 2, and the liver concentration of the compound was measured by LC/MS/MS.

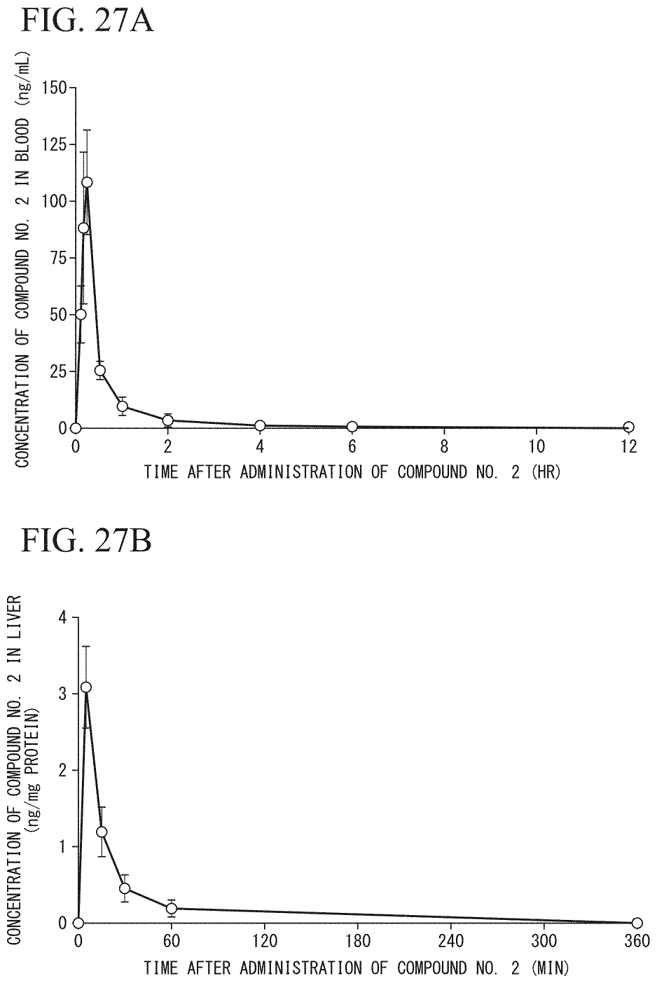

FIG. 27A is a graph showing the concentration of Compound No. 2 in blood. FIG. 27B is a graph showing the concentration of Compound No. 2 in liver. As a result, the concentration of Compound No. 2 in blood was highest at 15 minutes after administration, and the concentration of Compound No. 2 in liver was highest at 5 minutes after administration.

### Experimental Example 29

(Examination of Effect of Compound on Liver of DEN-Induced Hepatitis/Hepatoma Mouse Model)

Diethylnitrosamine (DEN) dissolved in water at a concentration of 80 mg/L as drinking water was administered in drinking water to ICR mice, thereby inducing chronic inflammation and hepatoma in the liver. In addition, a group in which 15 mg/kg of the compound of Example 10 (NS-3-086) was administered in drinking water from immediately after the start of administration of DEN in drinking water was prepared. As a control group, a group given tap water as drinking water was used.

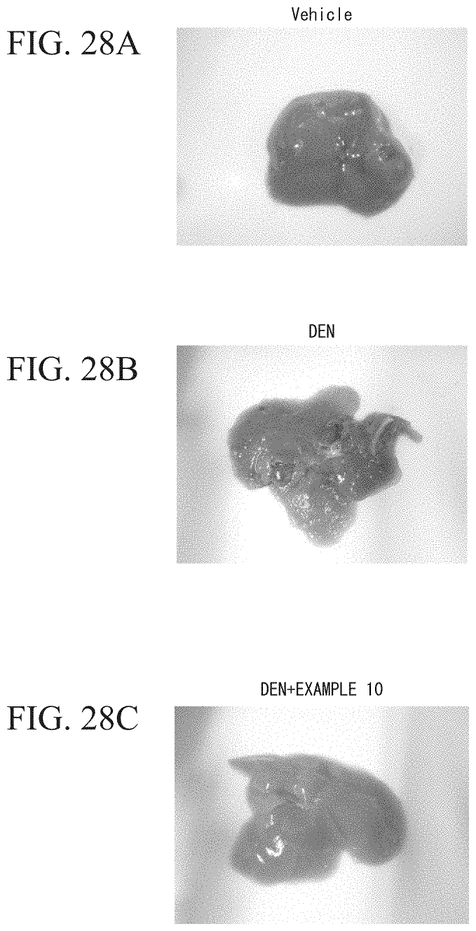

Subsequently, mice of each group were sacrificed 14 weeks after the start of administration of DEN in drinking water, and the liver was excised and analyzed. FIGS. 28A, 28B and 28C are photographs of the liver at 14 weeks after the start of administration of DEN in drinking water.

FIG. 28A is a representative photograph of a liver of a mouse of a control group (indicated as “VEHICLE” in the figure), FIG. 28B is a representative photograph of a liver of a mouse of a DEN drinking water group (indicated as “DEN” in the figure) and FIG. 28C is a representative photograph of a liver of a mouse of a group to which DEN and the compound of Example 10 were administered (indicated as “DEN+EXAMPLE 10” in the figure).

As a result, when compared with the control group, a large number of tumors were found, and hepatic hardening was observed in the liver of mice of the DEN drinking water group. On the other hand, tumor formation was inhibited and hepatic hardening was inhibited in the liver of mice of the group to which DEN and the compound of Example 10 were administered. Table 1 below shows the number of tumors in the liver of mice of each group.

TABLE 1

| | | | |
|---|---|---|---|
| Number of tumors | 0 | 1 | Large number |
| Control (n) | 0 | 0 | 0 |
| DEN drinking water group (n) | 0 | 0 | 6 |
| DEN + Example 10-administered group (n) | 3 | 3 | 0 |

The above results indicate that hepatic cancerization and hardening by DEN were inhibited by the administration of the compound of Example 10.

### INDUSTRIAL APPLICABILITY

According to the present invention, a novel anti-inflammatory drug can be provided.

1.-9. (canceled)

10. A method for treating or preventing an inflammation-related disease, including administering siRNA, shRNA, miRNA, ribozyme or antisense nucleic acid against G0S2, or a compound represented by General Formula (1) or a pharmaceutically acceptable salt thereof, or a solvate thereof to a patient or animal in need of treatment: 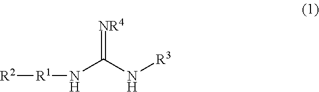
In General Formula (1), R¹ represents a single bond or an alkylene group having 1 to 3 carbon atoms, R² represents 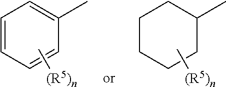
where
R⁵ represents a halogenated alkyl group having 1 to 3 carbon atoms and n represents an integer of 0 to 5,
R³ represents an alkyl group having 1 to 15 carbon atoms, and
R⁴ represents a hydrogen atom or a carboxylic acid ester group, and in the case where n is an integer of 2 or more, a plurality of R⁵s may be the same as or different from each other.

11. The method according to claim 10, wherein the inflammation-related disease is hepatitis or hepatoma.

12. A method for treating or preventing pain, including administering siRNA, shRNA, miRNA, ribozyme or antisense nucleic acid against G0S2, or a compound represented by General Formula (1) or a pharmaceutically acceptable salt thereof, or a solvate thereof to a patient or animal in need of treatment: 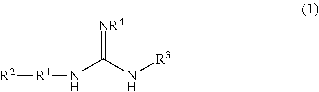
In General Formula (1), R¹ represents a single bond or an alkylene group having 1 to 3 carbon atoms, R² represents 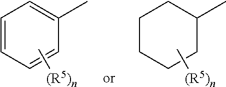
where
R⁵ represents a halogenated alkyl group having 1 to 3 carbon atoms and n represents an integer of 0 to 5,
R³ represents an alkyl group having 1 to 15 carbon atoms, and
R⁴ represents a hydrogen atom or a carboxylic acid ester group, and in the case where n is an integer of 2 or more, a plurality of R⁵s may be the same as or different from each other.
